(19) United States
(12) Patent Application Publication
(10) Pub. No.: US 2025/0242150 A1
(43) Pub. Date: Jul. 31, 2025

(54) ADDRESSABLE SERIAL ELECTRODE ARRAYS FOR NEUROSTIMULATION AND/OR RECORDING APPLICATIONS AND WEARABLE PATCH SYSTEM WITH ON-BOARD MOTION SENSING AND MAGNETICALLY ATTACHED DISPOSABLE FOR REHABILITATION AND PHYSICAL THERAPY APPLICATIONS

(71) Applicant: Neuvotion, Inc., Darien, CT (US)

(72) Inventors: Chad Bouton, Darien, CT (US); Sadegh Ebrahimi, Darien, CT (US); Joseph Toro, Darien, CT (US); John Leavitt, Darien, CT (US); Jan Niewiadomski, Darien, CT (US)

(21) Appl. No.: 18/854,837

(22) PCT Filed: Apr. 7, 2023
(86) PCT No.: PCT/US2023/017856

§ 371 Date: Oct. 7, 2024

Related U.S. Application Data
(60) Provisional application No. 63/328,349, filed on Apr. 7, 2022.

(51) Int. Cl. A61N 1/04; A61N 1/36

(52) U.S. Cl. CPC A61N 1/0484; A61N 1/0476; A61N 1/36031; A61N 1/36034; A61N 1/0452; A61N 1/0456

(57) ABSTRACT

The disclosure provides devices, systems, and methods for physical therapy. Electrical signals are applied to a subject via electrodes in a device worn on the body part receiving the therapy. The device can be operated remotely. The device can use artificial intelligence to evaluate and design physical therapy routines using the electrodes.

### CROSS-REFERENCE

This application claims the benefit of U.S. Provisional Application No. 63/328,349, filed Apr. 7, 2022, which is incorporated by reference herein in its entirety.

### BACKGROUND

Neurostimulation technology uses invasive or noninvasive approaches that apply electromagnetic energy to anatomical targets and induce neuromodulation of the corresponding neural circuitry for neurorehabilitation and other applications. Many neurostimulation systems, however, have a low number of electrodes which limits the number and precision in which muscles and neural targets can be activated and they are often not portable or wearable.

Wiring many electrodes in transcutaneous devices for neural or electrical stimulation and recording is challenging. Electrodes occupy much space. Routing large numbers of wires to sites of intended stimulation can be difficult and expensive. For wearable, transcutaneous applications, multi-layer flexible circuit boards bring the risk of decreased flexibility.

### SUMMARY

In some embodiments, the invention provides a device comprising: a first article, wherein the first article comprises 1) a first top surface, wherein the first top surface comprises a set of first connectors; and 2) a first bottom surface, wherein the first bottom surface comprises a plurality of electrodes; and a second article, wherein the second article comprises 1) a circuit board operably connected to the plurality of electrodes; 2) a second top surface, wherein the second top surface comprises a plurality of visualization aids, wherein each visualization aid of the plurality of visualization aids independently corresponds to one of the plurality of electrodes; and 3) a second bottom surface, wherein the second bottom surface comprises a set of second connectors, wherein each of the first connectors is independently configured to couple to one of the second connectors, wherein the first connectors and the second connectors are configured to form connections that hold the first article and the second article together when the first connectors are coupled to the second connectors, wherein when the first article and the second article are operably connected, the first article and the second article together form a wearable, wherein the wearable has a wearable size and a wearable shape adapted to fit on a human body part.

In some embodiments, the invention provides a device comprising: a) a first article, wherein the first article is disposable, wherein the first article comprises: 1) a bottom layer, wherein the bottom layer comprises: A) a hydrogel; and B) a plurality of electrodes in contact with the hydrogel, wherein the plurality of electrodes is connected in sequence, wherein the plurality of electrodes is flexible, wherein the plurality of electrodes are silver ink, wherein the plurality of electrodes are configured to stimulate muscle tissue and neural targets when the plurality of electrodes provide electrical stimulation to the muscle tissue and the neural targets; 2) a middle layer, wherein the middle layer comprises a dielectric material, wherein the middle layer is layered on top of the bottom layer, is in contact with the bottom layer, and is operably connected to the bottom layer; and 3) a top layer, wherein the top layer comprises a set of first connectors, wherein the first connectors are magnetic, wherein the top layer is layered on top of the middle layer, is in contact with the middle layer, and is operably connected to the middle layer, wherein the bottom layer has a bottom layer shape, the middle layer has a middle layer shape, and the top layer has a top layer shape, wherein when the bottom layer and the middle layer are operably connected, and the middle layer and the top layer are operably connected, then the bottom layer shape, the middle layer shape, and the top layer shape are substantially overlapping; b) a second article, wherein the second article is durable, wherein the second article comprises: 1) a second top surface, wherein the second top surface comprises a plurality of visualization aids, wherein each visualization aid of the plurality of visualization aids independently corresponds to one of the plurality of electrodes, wherein the plurality of visualizations aids are LEDs, wherein each visualization aid is configured to provide a visible signal when an electrode corresponding to a visualization aid emits an electrical signal; 2) a flexible circuit board comprising a conductor, the flexible circuit board being operably connected to the plurality of electrodes; and 3) a second bottom surface, wherein the second bottom surface comprises a set of second connectors, wherein the second connectors are magnetic, wherein each of the first connectors is independently configured to couple to one of the second connectors; and c) a control system operably connected to the second article, wherein the control system comprises: 1) a power supply; 2) a processor operably connected to the power supply and configured to operate the device; 3) a stimulator operably connected to the processor, operably connected to the power supply, and configured to send electrical stimulation to the flexible circuit board; 4) a wireless receiver operably connected to the processor and configured to receive instructions from a user for sending electrical stimulation to the flexible circuit board; 5) a memory system operably connected to the processor and configured to record information regarding use of the device; 6) a transmitter operably connected to the processor and configured to transmit wirelessly to a recipient external to the device a record of stimulation provided to a human body part in physical contact with the device and motion of the human body part in response to the stimulation; and 7) a motion detector operably connected to the processor and configured to detect motion of a human body part in physical contact with the device, wherein the power supply, the processor, the stimulator, the wireless receiver, the memory system, the transmitter, and the motion detector are in a common housing, wherein the first connectors and the second connectors are configured to form connections that hold the first article and the second article together when the first connectors are coupled to the second connectors, wherein when the first article and the second article are operably connected, the first article and the second article together form a wearable, wherein the wearable has a wearable size and a wearable shape adapted to fit on the human body part, wherein the first article has a first article shape, and the second article has a second article shape, wherein when the first article and the second article are operably connected, the first article shape and the second article shape are substantially overlapping, wherein the bottom layer has a surface area of at least 25 cm².

In some embodiments, the invention provides a method comprising: a) contacting a body part of a human subject with a plurality of electrodes, wherein the plurality of electrodes is operably connected to a flexible circuit board, wherein the body part has a shape; b) manipulating the flexible circuit board to conform substantially to the shape of the body part; c) operably connecting to the flexible circuit board a plurality of visualization aids, wherein each visualization aid of the plurality of visualization aids independently corresponds to one of the plurality of electrodes; and d) applying to the plurality of electrodes an electric current, whereupon the plurality of electrodes provide electrical stimulation to the body part.

In some embodiments, the invention provides a method comprising: a) contacting a body part of a human subject with a plurality of electrodes, wherein the plurality of electrodes is operably connected to a flexible circuit board, wherein the body part has a shape; b) manipulating the flexible circuit board to conform substantially to the shape of the body part; c) receiving instructions from a wireless user device for sending electrical stimulation to the flexible circuit board; and d) selecting at least a portion of the plurality of electrodes and applying to the portion of the plurality of electrodes an electric current, whereupon the portion of the plurality of electrodes provides the electrical stimulation to the body part.

In some embodiments, the invention provides an addressable electrode system, comprising: a power supply; and an article comprising: (i) a plurality of sequentially-connected electrodes; (ii) a plurality of indicators; and (iii) a plurality of switches, each switch independently comprising a first terminal, a second terminal, and a third terminal; wherein the first terminal of the switch is electrically coupled to the power supply; wherein the second terminal of the switch is electrically coupled to an indicator of the plurality of indicators; wherein the third terminal of the switch is electrically coupled to an electrode of the plurality of sequentially-connected electrodes; and wherein the switch is configured, when power is provided by the power supply to the first terminal of the switch, to provide electrical power simultaneously to the electrode and to the indicator.

### INCORPORATION BY REFERENCE

Each patent, publication, and non-patent literature cited in the application is hereby incorporated by reference in its entirety as if each was incorporated by reference individually.

### BRIEF DESCRIPTION OF THE DRAWINGS

FIG. 1 illustrates a schematic for implementing addressable electrodes used in a wearable system described herein.

FIG. 2 illustrates a set of configurations of electrodes described herein.

FIG. 3 illustrates a thin, lightweight, flexible, wearable system.

FIG. 4 illustrates a flexor wearable system on inner forearm for neurostimulation that evoke finger and wrist flexion movements.

FIG. 5 illustrates another view of the wearable system of FIG. 3.

FIG. 6 illustrates an exploded view of the wearable system of FIG. 3.

FIG. 7 illustrates a wireless control system and method for controlling neurostimulation via the wearable patch system described herein.

FIG. 8 illustrates a side view of the second article (electronic patch system) and first article (disposable system) described herein.

FIG. 9 illustrates a view of the disposable system described herein.

FIG. 10 illustrates electrode arrays of the disposable system described herein.

FIG. 11 illustrates a wrist worn embodiment of the wearable patch system described herein.

FIG. 12 illustrates a stimulation module described herein.

FIG. 13 illustrate a disposable electrode array configured to be attached to a patient's forearm and thumb.

FIGS. 14A-14E illustrate a disposable electrode array that is attached to a patient's forearm and thumb.

FIG. 15A-15C show the durable/reusable electronic patches that are attached to the disposable electrode arrays.

FIGS. 16A-16C illustrate the fixation of an assembly comprising a durable/reusable patch and corresponding disposable electrode array attached to the patient's forearm.

FIG. 17 illustrates a stimulator module attached to the wrist of the patient.

FIG. 18 illustrates the connections between the stimulator and the flexor and extensor patches and the thumb disposable electrode array via cables.

FIG. 19 illustrates a graphical user interface (GUI) of an application that allows the user to control the stimulation.

FIG. 20 illustrates a GUI of an application that allows the user to control manual stimulations.

FIG. 21 illustrates user's selection of multiple positions on the patch to perform electrical stimulation, each position being indicated by a LED.

FIG. 22 illustrates a GUI of an application that allows the user to control thumb stimulations.

FIG. 23 illustrates a GUI of an application for performing stimulation sequence.

### DETAILED DESCRIPTION

Neurostimulation can elicit muscle contractions or modulate nerve, spinal cord or spinal cord roots using electric impulses. A device can generate and deliver impulses through electrodes to the skin in proximity to muscles and neural targets being stimulated. Electrodes can be pads adhering to or contacting the skin. The provided impulses can evoke action potentials in the nerves or cause muscles to contract. Neurostimulation is used, for example, in rehabilitation (e.g., in physical or occupational therapy), for strength training, and for evaluating neural function. For example, Neurostimulation can be used to address muscle atrophy, improve muscle weakness, or modulate spinal cord pathways to promote rehabilitation of movement or sensory function.

Neurostimulation technology provides positive outcomes in neurorehabilitation and physical therapy applications including post stroke and injury cases. These outcomes include improved movement, sensation, and independence for those living with impairment. A neurostimulation systems with a small number of electrodes limits the number and precision in which muscles and neural targets can be activated and are often not portable or wearable. Furthermore, systems/devices that do not include on-board sensing or microprocessors that can execute AI or machine learning algorithms to recognize user intentions can be less intuitive and functional for assistive and rehabilitative applications. User-driven neurostimulation to facilitate goal-oriented rehabilitative tasks is much more effective than passive stimulation is.

Another challenge in neurostimulation devices and systems is that manual placement of electrodes is difficult and time consuming. This challenge encumbers location and stimulation of smaller muscles, such as those that control hands and fingers and also spinal cord dorsal root targets, which are important in promoting plasticity and recovery.

The present disclosure provides systems and methods that resolves the aforementioned challenges in neurostimulation. Disclosed herein is a system that provides a wearable, user-driven device with dynamic (electronically configurable) electrical stimulation to a target body area. The system can be, for example, a thin, lightweight, flexible, wearable patch system. Non-limiting examples of body areas to be treated with the system include feet, lower legs, upper legs, hips, buttocks, lower back, upper back, the abdominal region, the chest, shoulders, upper arms, forearms, wrists, hands, base of head, head (e.g., top of head, forehead), and the spinal cord. An example device or system comprises one or more articles, for example, patches. The articles are placed, for example, behind, in front of, outside, or inside areas of the body. One or more of the articles in a device or system can comprise a set of electrodes (e.g., arranged in an array, sequence, or matrix) to contact the skin and provide electrical stimulation to the underlying muscles. In some embodiments, the articles are communicatively coupled to a unit providing the electrical signals. In some embodiments, the articles are communicatively coupled to a microprocessor configured to select electrical signals to stimulate particular muscles, nerves, or spinal roots corresponding to particular regions of the body. In some embodiments, the systems and methods disclosed herein are used for electrical muscle stimulation (EMS), electrocorticography, or electroencephalography.

A device or system can comprise a plurality of electrodes. In some embodiments, the electrodes are daisy-chained (e.g., wired together in a sequence) and communicatively coupled to indicators or control systems via switches (e.g., solid state relays).

In some embodiments, a device or system includes a machine learning software module. The machine learning software module uses one or more machine learning algorithms to analyze motion of a body part being provided electrical stimulation. The machine learning algorithms can be trained through the process of collecting data related to the electrical signals provided to the muscles and/or neural targets (e.g., spatial information regarding which electrodes on the patch provided signals), motion of the body part, or both, to predict motion of the body part. Non-limiting examples of motion include extension, flexion, angular motion (e.g., rotation), clenching (e.g., of a fist), and relaxation of one or more body parts. In some embodiments, the motion is exercise (e.g., a press, curl, extension, squat, throw, or hip hinge).

In some embodiments, the wearable system is configured to interoperate with a brain-computer interface (BCI). A device (e.g., headgear) can derive electrical signals from the brain and provide the signals to the wearable system. The wearable system can contain a processor configured to execute instructions based at least in part on the signals to stimulate particular electrodes. An algorithm (e.g., a machine learning algorithm) can interpret the signals from the brain to determine body movements associated with the signals. Based on this interpretation, a controller can drive the electrodes to stimulate these body movements. Subjects with diseases, injuries, or conditions preventing the brain from implementing motor functions can use the wearable system disclosed herein to regain these motor functions.

### Wearable System

Devices and systems disclosed herein include wearables, for example, articles, patches, textiles, fabrics, and other wearable systems. In some embodiments, wearable systems (e.g., wearable patch systems) disclosed herein can be configured to be incorporated into other textile articles, such as medical articles or clothing (e.g., casts, wrappings, braces, bandages, gowns, shirts, pants, undergarments, watches, bracelets, headbands, headgear, or athletic wear).

The wearable system can be configured to be worn by a subject. The subject can be a human subject or a non-human subject. For example, the subject can be a mammalian subject, such as a dog, cat, horse, cow, pig, sheep, goat, rabbit, elephant, camelid (e.g., camel, alpaca, or llama), mouse, hamster, guinea pig, ferret, buffalo, donkey, deer, or monkey. The wearable system can be worn by a bird or reptile, for example, a chicken, duck, goose, emu, eagle, alligator, or crocodile.

In some embodiments, the system comprises a first article, which is configured to provide electrical signals to a body part, and a second article, which is configured to provide indications with respect to where on the body the electrical signals are being provided.

In some embodiments, the electrical signals are configured for neurostimulation (e.g., muscle tissue or neural target stimulation). For example, an electrical signal provided by the system disclosed herein can elicit muscle contraction. The electrical signal is provided by an electrode placed on the surface of the skin in proximity to the muscle being stimulated. In some embodiments, the system provides therapy by causing repeated muscle contractions and improving blood flow to stimulate repair of injured muscles. Repeated cycles of contraction and relaxation can also improve muscle strength. The system disclosed herein can be used for many types of electrical muscle stimulation, including transcutaneous electrical nerve stimulation (TENS), electrical muscle stimulation (EMS), electrical stimulation for tissue repair (ESTR), interferential current (IFC), neuromuscular electrical stimulation (NMES), functional electrical stimulation (FES), spinal cord stimulation (SCS), and iontophoresis.

In some embodiments, the first article has a first article shape. The second article has a second article shape. When the first article and the second article are operably connected, the first article shape and the second article shape can be substantially overlapping. In some embodiments, the second article shape can be larger than the first article shape is. The first article shape and/or the second article shape can conform to at least a portion of a body part of a subject (e.g., a human subject). For example, the first article shape and/or the second article shape can bend, flex, or fold to provide sufficient electrical and/or physical contact or coupling with a portion of the body. The first article can be disposable. The second article can be durable. The first article can comprise a bottom layer, a middle layer, and a top layer. The middle layer can be layered on top of the bottom layer, can be in contact with the bottom layer, and can be operably connected to the bottom layer. The top layer can be layered on top of the middle layer, can be in contact with the middle layer, and can be operably connected to the middle layer.

In some embodiments, the first article comprises a plurality of electrodes and a layer of hydrogel.

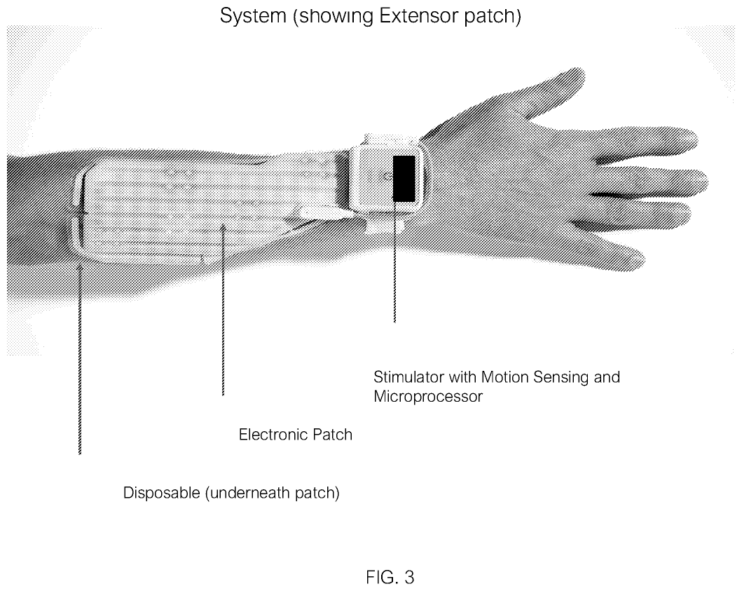

FIG. 3 illustrates a thin, lightweight, flexible, wearable system described herein. The wearable system can comprise a first article and a second article, for example, patches or textiles, which can be physically and/or communicatively and/or electrically coupled. The second article can be disposed above the first article, so that a portion contacts the skin of a subject and that a surface of the first article is visible. In some embodiments, the first article comprises a disposable system. The disposable system comprises, for example, a plurality of disposable magnetic connectors for coupling the first article to the second article. The magnetic conductors can be conductive and thus configured to pass electrical current through the wearable system. Thus, the magnetic connectors can provide both magnetic and electrical coupling. In some embodiments, the second article comprises an electronic patch system.

An electronic patch system can comprise a plurality of patches. The patches can be made from darts of textiles (e.g., fiber-based materials such as fabrics or cloths). A patch can comprise at least one indicator (or visualization aid). A plurality of indicators can be disposed or arranged on the second article. An indicator can be placed above (e.g., disposed on top of) or underneath a patch, such that the indicator and the patch correspond. The patches (and, thus, indicators) can be arranged in an array (e.g., a square or rectangular array, or any shape suitable for the intended body part). The indicators can comprise audio, visual, haptic, or audiovisual indicators, or a combination thereof. For example, in some embodiments, the indicators produce light and/or sound to alert a subject or user where and at what time an electrical signal is being provided to a subject's body through one or more electrodes of the wearable patch system. An indicator can comprise, for example, a light-emitting diode (LED). An indicator of the second article can correspond to one or more electrodes of the second article. The indicator can produce light, or a visible signal, simultaneously when the corresponding electrode emits a signal. For example, the one or more electrodes of the first article can be placed directly underneath a particular indicator of the second article. When neurostimulation is provided by a plurality of electrodes at different spatial regions of the patch system simultaneously, indicators of corresponding regions can provide audiovisual signals (e.g., light up) simultaneously. In this manner, patterns of stimulation of muscle tissue and neural targets from different regions of the body underneath the patch can be displayed on the surface of the second article.

The electronic patch system can comprise a stimulator module or a control system. The stimulator module can control the provision of electrical signals to the body part and monitor and record data resulting from neurostimulation caused by the provided electrical signals. The stimulator module may comprise a plurality of modular components within a housing.

The stimulator module can comprise a processing unit or processor (e.g., a microprocessor). In some embodiments, the processor is external and operably coupled to the stimulator module. The processor can receive instructions (e.g., provided by a health care professional or provided automatically by a computer program, for instance, algorithmically) indicating a set of electrodes on the first article of the wearable system for providing neurostimulation to a region of the body overlaid by the wearable system. The instructions can also comprise a corresponding set of indicators to provide audio, visual, haptic, audiovisual signals, or a combination thereof, to a user (e.g., a subject or wearer of the patch, a supervisor, or a health professional). The processor can determine which patch (and indicator) corresponds to which set of electrodes using, for example, a personality resistor.

The processor can implement instructions configured to control the neurostimulation. For example, the control instructions can define which electrodes are to provide the stimulation and at what times. The control instructions can provide a pattern of electrical stimulation. For example, the control instructions can direct electrical pulses to be provided to particular muscles or neural targets periodically (e.g., every second, five seconds, 10 seconds, 20 seconds, or 30 seconds).

The processor can implement instructions defining characteristics of the electrical impulses. For example, an electrical impulse can have a frequency between 1 and 10 Hz, 11 and 20 Hz, 21 and 30 Hz, 31 and 40 Hz, 41 and 50 Hz, 51 and 60 Hz, or 61 and 70 Hz. The impulse duration can be between 50 and 100 microseconds (μs), between 100 and 150 μs, or between 150 and 200 μs. The stimulation duration can be between 0 and 5 seconds, between 5 seconds and 10 seconds, between 10 seconds and 20 seconds, or between 20 seconds and 30 seconds.

The instructions can be received from an external computing device (e.g., from a mobile computing device) connected via a wired or wireless network. The network can be, for example, an Ethernet network, a Wi-Fi network, or a Bluetooth network.

The stimulator module can comprise a motion sensing unit, which may comprise an inertial measurement unit (IMU). The IMU can comprise an inertial sensing integrated circuit (IC), which has, for example, six or nine degrees of freedom (DOF) to sense different types of rotational movement about a set of axes (e.g., surge, heave, and sway) and translational movement about a set of axes (e.g., roll, pitch, and yaw). The inertial measurement unit can comprise, for example, one or more accelerometers, gyroscopes, and/or magnetometers.

The stimulator module can comprise a machine learning software module, which provides one or more machine learning models that can be configured to process measurements made from the IMU, electrical signals provided through the electrodes, or instructions for providing the electrical signals, to predict motion of the part of the body on which the patch is worn. In some cases, the machine learning software module is external to the stimulator module. For example, the machine learning software module can be located on a server that is communicatively coupled to the stimulator module (e.g., over a wired or wireless network).

In some embodiments, the stimulator module (or control system) or processor are operably connected to a power supply. In some embodiments, the power supply and stimulator module are in a common housing. The power supply can comprise a battery. The power supply can comprise an AC adapter. In some embodiments, the stimulator comprises electrical components sufficient to derive voltage and power from mains power, when plugged into a wall socket.

The stimulator module can further comprise a memory system operably connected to the processor and configured to record information regarding use of the wearable patch system. The memory system can additionally comprise one or more machine learning models that can be executed by the processor on data recorded in the memory.

The stimulator module can further comprise a transmitter operably connected to the processor and configured to transmit wirelessly to a recipient external to the device the information regarding the use of the device. The transmitter can transmit information via radio waves, such as over a Wi-Fi network or a Bluetooth network.

The disposable system can comprise a dielectric disposed above a plurality of electrodes. A dielectric can comprise any material that can act as an electrical insulator and that can be polarized by an applied electric field, such as porcelain, glass, mica, metal oxide, or plastic. Example plastics can comprise thermoplastic polymers such as polyvinyl chloride (PVC or vinyl) or polyethylene terephthalate (PET). The electrodes can be flexible electrodes. An electrode can comprise a paste or ink made of one or more conductive materials or substances (e.g., metals, for example, silver or gold). In some embodiments, the electrodes are coated with carbon nanotubes.

FIG. 3 illustrates a device worn on the forearm. The example device includes two sets of the first article and second article communicatively and physically coupled together, where a first set is configured to be worn on the back of the forearm and a second set is configured to be worn on the inside of the forearm. Wearable devices on the back of and inside of the forearm can provide electrical stimulation to muscles and neural targets configured to evoke wrist, hand, and finger movements.

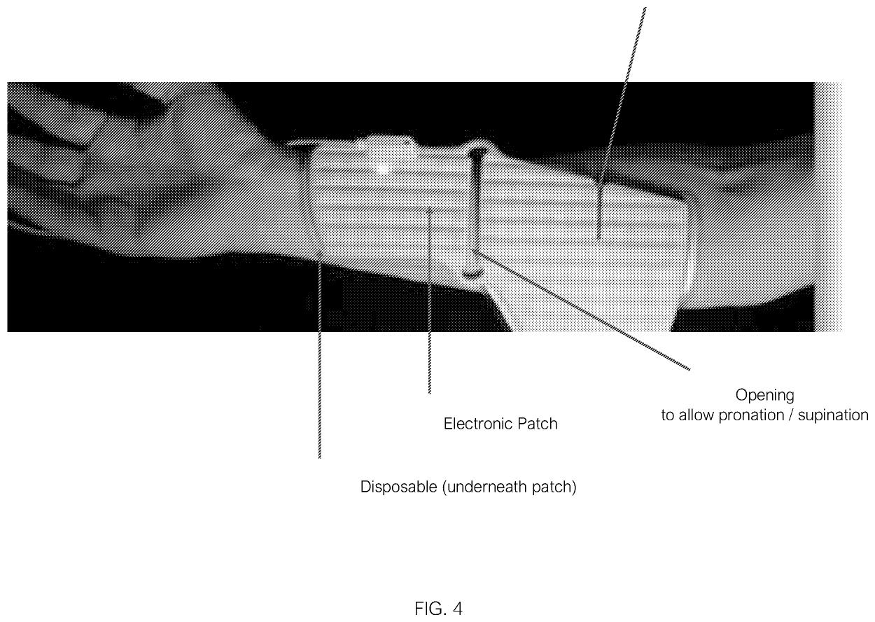

FIG. 4 illustrates a flexor wearable system on the inner forearm for stimulating muscles and neural targets that evoke finger and wrist flexion type movements described herein. Like in FIG. 3, the embodiment of FIG. 4 includes an electronic patch system and a disposable system. The flexor patch system comprises an opening to allow pronation and supination of the wrist. In some cases, an identical wearable system can be worn on the other side of the arm to stimulate additional muscles and neural targets that perform complex movements.

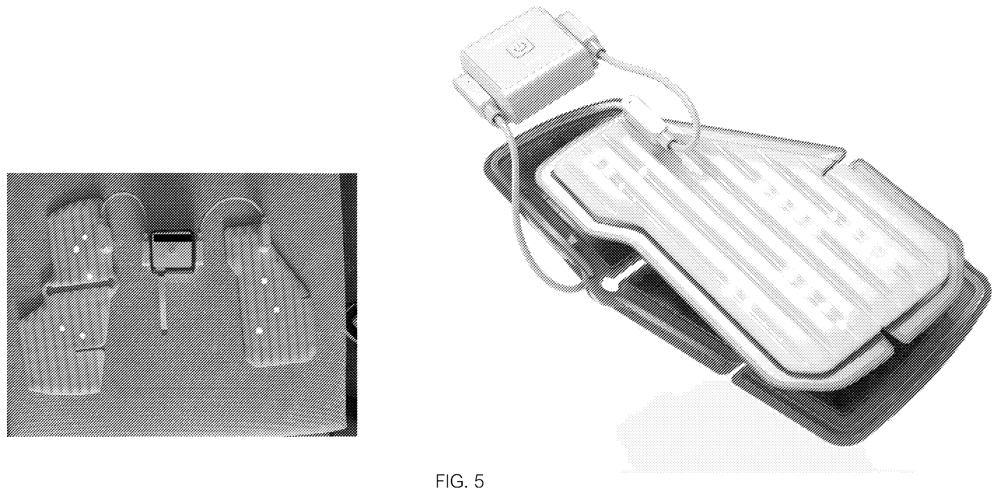

FIG. 5 illustrates another view of the wearable system of FIG. 3. FIG. 5 shows a first wearable system to be placed inside the forearm and a second wearable system to be placed outside the forearm. FIG. 5 additionally illustrates a view of the first wearable patch system, the second wearable patch system, and the stimulator module together side-by-side.

FIG. 6 illustrates an exploded view of the wearable system described herein. FIG. 6 illustrates the second article (electronic patch system), stimulator module, first article (disposable system), and cabling. Disposables can have conductor paste or ink (e.g., silver ink) electrodes on one side and magnetic connectors on the opposite side. This arrangement can create mechanical and electrical connections to the durable/reusable electronic patch system. The stimulator (wrist worn) can include a cloth or textile strap including an adhesive unit, such as Velcro or magnetic snaps. In some embodiments, forearm straps can be included (not shown) to wrap around the device when worn on the user's arm. These straps can keep all the elements of the wearable patch system in good contact. These straps can be textile elastic and can have, for example, Velcro or magnetic snap connections.

In some embodiments, at least two stimulators can be connected to a plurality of patches. For example, the wearable system can comprise a first wearable patch system, a second wearable patch, and a first stimulator module, as illustrated in FIG. 5. The first and second wearable patch systems can be applied to a patient's forearm and controlled by the first stimulator module connected thereto. The wearable system can further comprise a third wearable patch system connected with a second stimulator module. For example, the third wearable patch can have a small size and be applied to the patient's neck and when controlled by the second stimulator, can provide neurostimulation to the patient's spinal cord. When a patient wears the forearm patches and patch in proximity to the spinal cord, both of which generate neurostimulation, the efficacy of the stimulation can exceed that of the forearm muscle stimulation alone.

In some embodiments, a wireless control system can be used to control the neurostimulation via one or more of the stimulators. When a plurality of patches is connected to the stimulator, a user can use the wireless control system to control the neurostimulation. The user can send wireless signals to the stimulator, which in turn controls the provision of electrical signals from the designated patches to the body part and monitor and record data resulting from neurostimulation caused by the provided electrical signals. For example, the stimulator can control the two forearm patches to generate neurostimulation in a given patten, and the neck patch in another given pattern. The stimulator can control simultaneous stimulations from a plurality of patches. Alternatively, the stimulator can control the stimulations from different patches to occur in any configuration (e.g., sequence or spatial arrangement) that a user provides. The user can save the configurations of the generated neurostimulations from different patches and the patterns thereof for future use, without going through manual settings repeatedly.

In some embodiments, the wearable patch can comprise a transmitter operably connected to a processor within the patch and configured to transmit wirelessly to other wearable patches. The transmitter can transmit information via radio waves, such as over a Wi-Fi network or a Bluetooth network. Hence, the wearable patch can communicate with and control other patches. For example, the forearm and neck patches can comprise a transmitter operably connected to a processor within the patch. When the forearm patches generate neurostimulation, the forearm patches can automatically control the neck patch to generate neurostimulation. The double stimulation can significantly improve the efficacy of rehabilitation of movement or sensory function and strength training. Additionally, stimulating the spinal cord in this manner can result in greater plasticity and/or adaptability of the body, providing for improved rehabilitation.

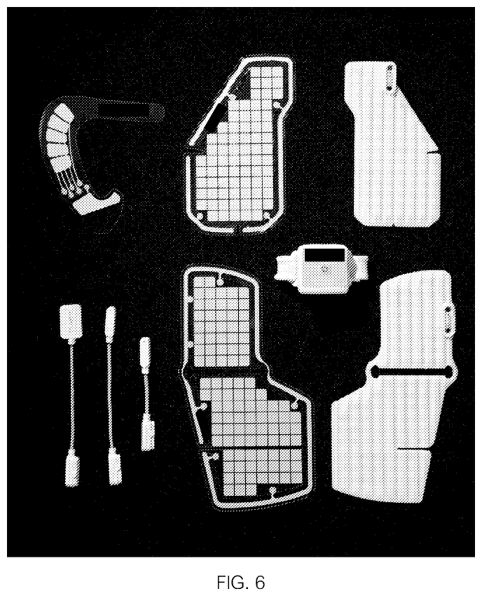

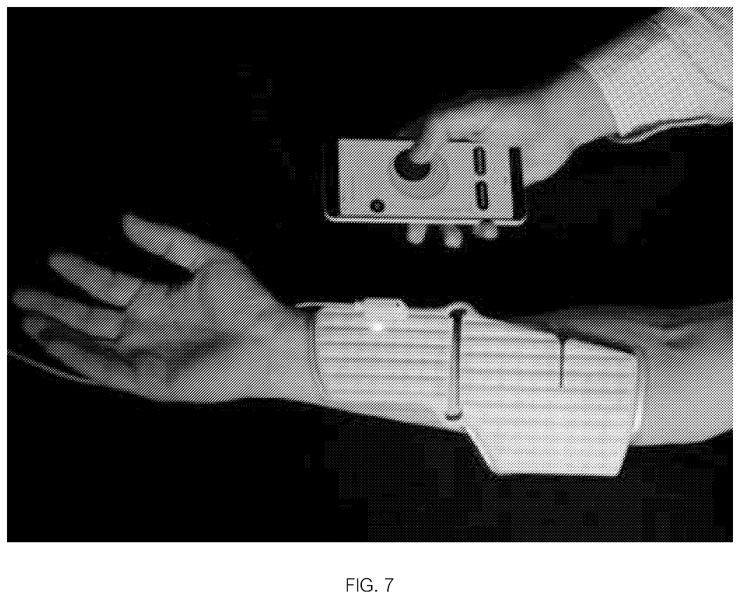

FIG. 7 illustrates a wireless control system and method for controlling neurostimulation via the wearable system. A telecommunications system or mobile device can include an application for configuring, controlling, and/or monitoring neurostimulation from the wearable patch system (e.g., during physical therapy, occupational therapy, athletic coaching, or medical rehabilitation), and collecting data related to electrical signals provided to the skin and movements of body parts responsive to the provided electrical signals. The data can be provided to external servers, e.g., cloud servers, for further analysis (e.g., machine learning analysis).

The telecommunication system (e.g., mobile device) can be configured to provide analysis of a performance of a human subject using the wearable system in therapy and to determine a proposed therapy regimen based on the analysis. For example, the telecommunication system can process, or provide for processing of, motion data of a human subject, to determine whether the subject is performing an exercise properly, or to determine a person's ability to perform a complex motion or sequence of motions. This analysis can be performed using machine learning or statistical methods. Additionally, the telecommunications system can analyze motions of a human subject undergoing therapy that are induced by providing neurostimulation, and compare the motions to motions performed by a healthy individual. Features of the analyzed motion can indicate a particular condition requiring a particular therapy regimen. For example, the analysis can determine that a person cannot effectively grip an object, or cannot perform an exercise with a proper range of motion. The system could recommend therapies to correct these conditions. The proposed therapy regimen can comprise activation of the electrodes in a pattern that is determined to be likely to provide a physical therapy improvement based on the analysis.

The application can be a desktop application or a mobile application (e.g., an ANDROID® application or an iOS® application). The application for configuring, controlling, and/or monitoring the neurostimulation facilitates configuration, control, or monitoring via a user interface. A non-limiting example of a user interface is a graphical user interface (GUI). In some embodiments, the GUI comprises a layout. The layout can be two-dimensional. In some embodiments, a layout is three-dimensional (for example, when multiple wearables are positioned in different locations on the body, and electrodes can be addressed using a three-dimensional coordinate system instead of two). A layout can comprise a grid. The grid can use Cartesian or polar coordinates. In some embodiments, a three-dimensional grid uses Cartesian, cylindrical, or spherical coordinates. Points on the grid can comprise two individual electrodes within an arrangement of electrodes. The arrangement of electrodes can be incorporated into or embedded within an article (e.g., the first article of the wearable system).

Electrical impulses or neurostimulation impulses can be initiated by a gesture provided to the UI. The gesture can comprise physical contact (e.g., with a body of a subject or with an instrument or tool). The gesture can be produced by a human hand or by one or more digits of the human hand. In some embodiments, the gesture can be produced by another body part. For example, a gesture can be produced by a movement of one or both eyes (which can include winking or blinking), in cases where a subject is paralyzed or paraplegic. The gesture can comprise a touch. Touching the UI on a point on the user interface (e.g., grid) can generate a control signal that can be used to provide neurostimulation or electrical stimulation to a subject via an electrode contacting the subject and corresponding to the point on the user interface. The touch can comprise one or more taps. Tapping a point on the grid can produce an electrical impulse that has the same duration as the tap. Tapping a point on the GUI repeatedly can produce repeated impulses (e.g., as a time series sequence) provided by the corresponding electrode. Tapping multiple points on the GUI can produce spatially distributed impulses on the grid. A touch can also comprise a press (e.g., of a digit or stylus) on the UI. The impulse can last as long as the duration of the press. A touch can comprise sliding or dragging a digit or stylus while pressed on the surface of the device providing the UI. As the digit or stylus is slid or dragged over points on the UI, corresponding electrodes can be stimulated or activated (e.g., transiently) to produce impulses. A gesture can comprise a combination of taps, presses, and drags (e.g., when multiple fingers or hands are used).

In an example, a user presses down on the screen in a location, and slides the finger across the screen (e.g., in a shape or pattern, such as a line, circle, or figure-8). As the finger slides across the screen, corresponding electrodes activate transiently, mirroring the pattern of movement on the finger. In this manner, dynamic electrical stimulation is provided to the subject's body mimicking the finger movement, such that the electrical impulses can be considered to “move” throughout the portion of the body covered by the electrode array.

The gestures can provide for multiple electrodes to be stimulated simultaneously (e.g., in a spatial configuration). For example, multiple fingers, digits, or instruments (e.g., styluses) can tap, press, slide, and/or drag multiple points on the UI.

A gesture can be performed or provided using an input/output (I/O) device, such as a keyboard, mouse (e.g., by clicking, or clicking and dragging), trackball, microphone (e.g., audio-controlled or speech-controlled neurostimulation), motion capture device (e.g., a camera that can monitor gestures that do not involve physical contact with the device comprising the UI), joystick, or other I/O device. The UI can allow a user to enter a sequence of commands (e.g., a script) or upload a computer program comprising instructions for neurostimulation with particular electrodes of the array.

The UI can provide for control of multiple electrode arrays (e.g., incorporated into multiple wearable patches). The UI can accomplish this control using a split screen, where each split presents one grid comprising one distinct electrode array. In some embodiments, the UI can display multiple grids (e.g., two, three, four, five, or ten grids) simultaneously. In some embodiments, multiple UI windows can be displayed and interacted with on multiple screens or monitors.

The control signal can be provided to one or more stimulation modules. The control signal can be provided over a network (e.g., a Bluetooth, Wi-Fi, or wired network (e.g, Ethernet). The control signal can encode information relating to a type of gesture performed and/or spatial position information relating to the gesture (e.g., for providing a spatial pattern of recommended neurostimulation impulses for the subject). The control signal can provide this information as instructions that, when executed by the processor, connects corresponding electrodes of the electrode array to power (e.g., via a power supply or power source, such as a battery or alternating current (A/C) adapter) using switches connected to and corresponding with the electrodes, such that electrical current is provided to the electrodes. For example, the instructions can place a first plurality of switches in an “ON” position and a second (non-overlapping) plurality of switches in an “OFF” position, allowing neurostimulation to be produced to the electrodes connected to “ON” switches. In this manner, the current provided to the electrodes provides to the subject the spatial pattern of recommended neurostimulation impulses corresponding to the gesture provided to the UI.

Non-limiting examples of the mobile device or telecommunications system include a cell phone, smartphone, personal digital assistant (PDA), laptop computer, desktop computer, and other types of computing device.

The mobile device can be used to provide dynamic (e.g., electronically-moveable) stimulation to a subject wearing the wearable system. The dynamic stimulation can use a touch pad or screen area on mobile device to allow rapid motor point mapping on a body part (e.g., a forearm). This technique can comprise, for example, an operator (e.g., the subject wearing the patch system or a health care provider) sliding a finger or stylus on a touch screen control object or area to change where on the subject body part the stimulation occurs (i.e., which electrode(s)) on the selected patch. A person can repeatedly tap particular sections of the stylus or grid to provide electrical impulses repeatedly to particular regions covered by the wearable system. An indicator (e.g., an LED) indicates the current stimulation (activated electrode) location on the electronic patch system of the wearable patch system.

The user interface allows modification of the impulses provided to the subject. For example, the user interface allows modification of the frequency of the impulses, the impulse duration, and/or the stimulation duration. These characteristics can be dynamically changed. For example, impulse frequency and/or impulse duration can be manually changed throughout a stimulation. In some cases, patterns of changing impulse frequencies and/or impulse durations can be programmed using the user interface.

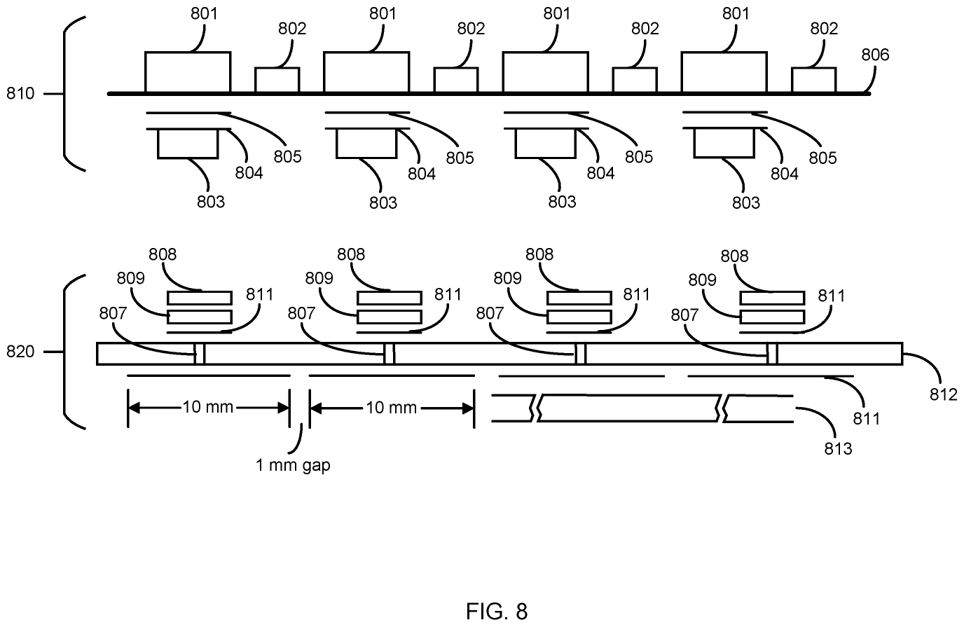

FIG. 8 illustrates a side view of the second article (electronic patch system) and first article (disposable system) described herein. The second article includes indicators 802 (e.g., LEDs) interspersed with solid state relays 801. The LEDs are disposed above a flex circuit or printed circuit board (PCB) 806. The underside of the layer 810 can be an electrical connection system comprising metal (e.g., gold) plated contacts 805 connected to magnets 803. The first article can comprise a vinyl surface 812 used as a dielectric, with ferromagnetic discs 808 and foam with conductive adhesive 809 disposed above the surface, and silver ink electrodes 811 adhered to the skin with hydrogel 820. In some embodiments, alternative adhesives and conductive materials for electrodes can be used.

Removal of hydrogel from metal electrodes can be difficult, time consuming, and often leaves unwanted residue. The disclosed system can solve this issue using a magnetic connection by creating a multi-layer disposable with a hydrogel layer (on the bottom/skin side), silver ink to form flexible electrodes, a dielectric (e.g., vinyl or polyethylene terephthalate (PET)) layer, and a magnetic (and electrical) connection layer on top to attach to the durable electronic patch system. The disposable system can then be removed and discarded after a set number of uses with no residue left on the electronic patch system.

The electronic patch system can be made of a flexible circuit board construction with, for example, gold plated copper as the conductor carrying stimulation energy. Plating, for example, nickel-copper-nickel, can be bonded to the flexible circuit conductors using, for example, conductive epoxy or conductive pressure sensitive adhesive (or known as conductive transfer tape or conductive double-sided tape (e.g., 3M 9711S)).

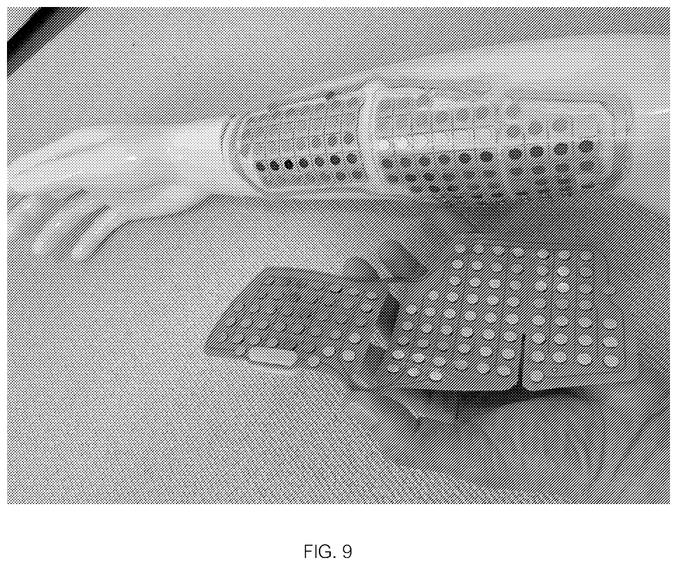

FIG. 9 illustrates a view of the disposable system described herein. The disposable system can comprise a magnetic attachment and an electrode array. The magnetic attachment can automatically align the electronic patch system with electrical contact points on the disposable electrode array. The electrode array can comprise a plurality of electrodes that are configured to provide electrical signals to human skin.

FIG. 10 illustrates electrode arrays of the disposable system described herein. The electrode arrays can be shaped to accommodate various body parts. The electrode arrays can also conform to a body part or a portion of a body part (e.g., by curving or folding), while maintaining mechanical contact between the body and every electrode (while allowing lateral sliding when a body part, muscle flexes or neural targets under electrode array) and additionally maintaining electrical contact between the body and the electrodes, allowing transmission of stimulation current to the electrodes through conductive plating over the magnets in the magnetic attachment. FIG. 10 additionally illustrates a thumb electrode array geometry, which can cover a thumb rotator plus transverse and oblique adductors.

In some embodiments, the electrodes are arranged in a square or rectangular arrangement. This arrangement can be used for neuromuscular electrical stimulation (NMES) or transcutaneous electrical nerve stimulation (TENS). The stimulator module can support a wide variety of waveforms for both NMES or TENS mode.

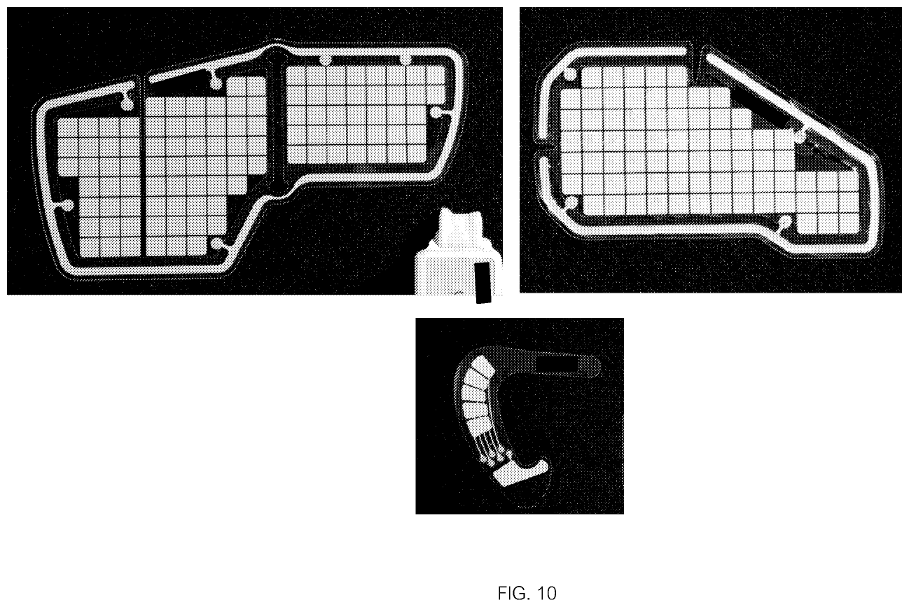

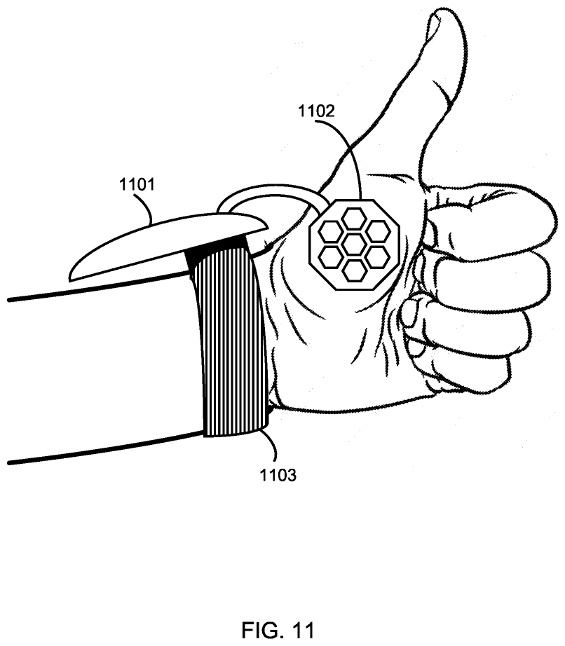

FIG. 11 illustrates a wrist-worn embodiment of a wearable system. The wrist worn embodiment can include, for example, a stimulator unit 1101, an electrode array with hydrogel 1102, and a wrist strap 1103.

FIG. 12 illustrates an example of a stimulation module. The stimulation module can include a microprocessor, inertial motion sensor (IMU), battery, accessible electrodes, and stimulation circuit. The embodiment of FIG. 12 is configured to be worn on the wrist, but in other embodiments, the stimulation module is worn on another part of the body. In some embodiments, the stimulation module is embedded in the patch. The wearable patch system can also be linked, for example, wirelessly, to a brain-computer interface (BCI) system.

The stimulation module can be used in applications including upper extremity stimulation, lower extremity stimulation (both for quadriceps and dorsal flexion), and cervical stimulation (base of neck).

The microprocessor can also store stimulation sequences (e.g., sequences of electrical pulses provided from the electrodes to the muscles or neural targets). Particular stimulation sequences can evoke particular movement sequences. For example, a stimulation pattern can cause opening of a user's hand for a few seconds, allowing the user to position around an object. Then a stimulation pattern causing the user to close the hand around the object can be applied. This configuration allows that a single command triggers a useful sequence.

The microprocessor can process stimulation multiplexing. Electrical stimulation pulses can be nested or interlaced in the time domain to allow for multiple muscles or nerve targets to be stimulated simultaneously but where individual pulses do not coincide.

### Addressable Electrodes

Devices and systems described herein comprise, for example, a plurality of electrodes. Characteristics of these electrodes include a wiring configuration, for example, in a daisy-chain or sequential fashion and communicative coupling to indicators or control systems via switches (e.g., solid-state relays). In some embodiments, the electrodes are configured to be placed on the body (e.g., contacting the skin) or in some embodiments are implantable.

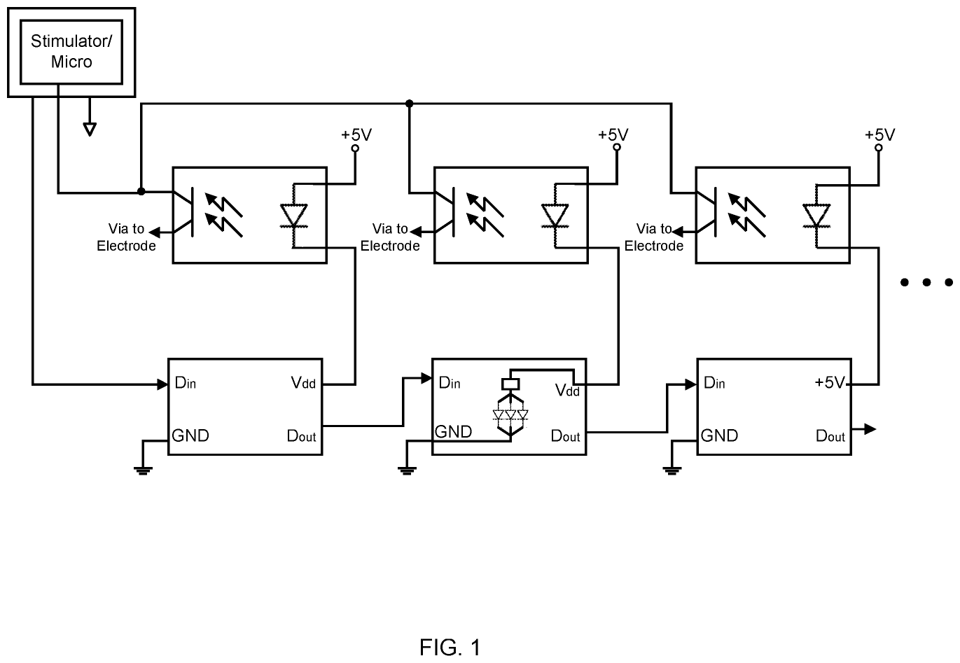

FIG. 1 illustrates a schematic for implementing electrodes used in a system described herein.

Electrode or electrode pairs 101 can be daisy-chained (e.g., wired together in sequence) to lessen the amount of wiring needed. Suitable electrode configurations include an anode and cathode, multiphase electrodes, and return electrodes. Doubling the circuit can allow an electrode to be either an anode or a cathode. The electrodes are connected to indicators (e.g., LEDs) or controllers 103 using switches 104 (e.g., solid-state relays, metal-oxide-semiconductor field effect transistors (MOSFETs), or other switch topologies). In some embodiments, the addressable indicators and/or controllers 103 and switches 104 are integrated into a single integrated circuit (IC), further reducing the size and complexity of the architecture.

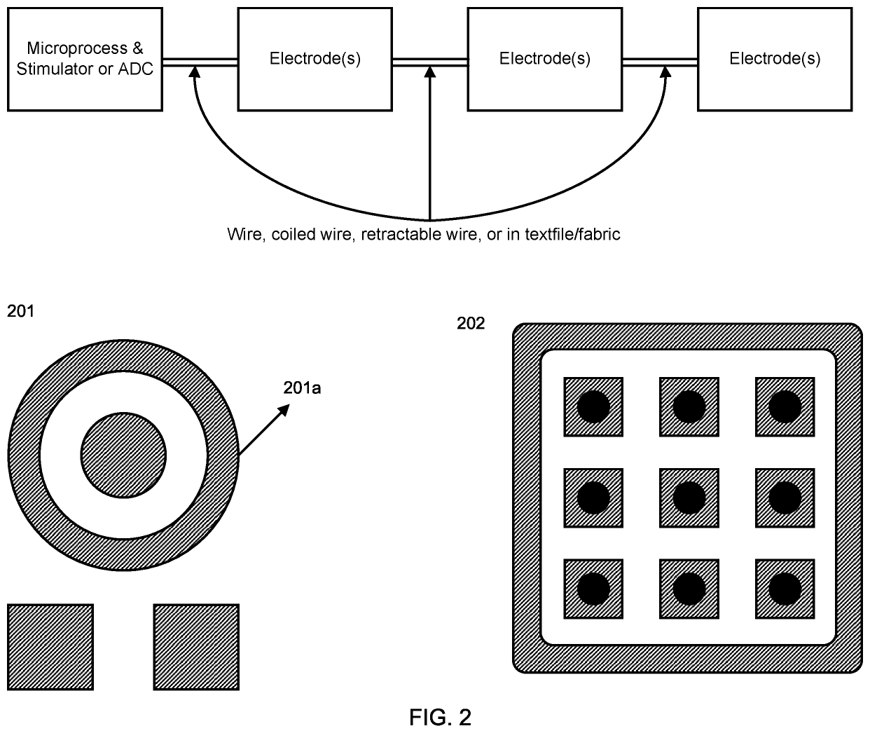

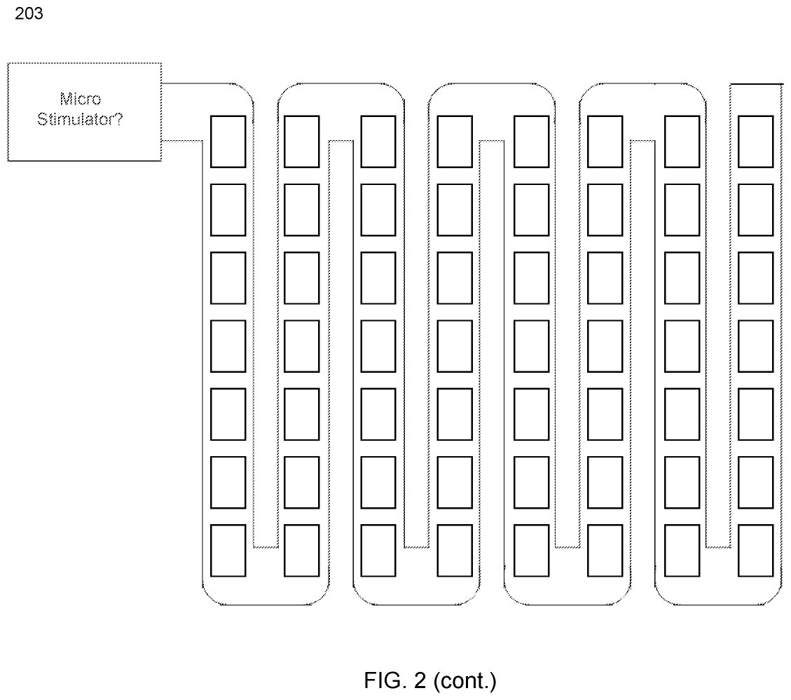

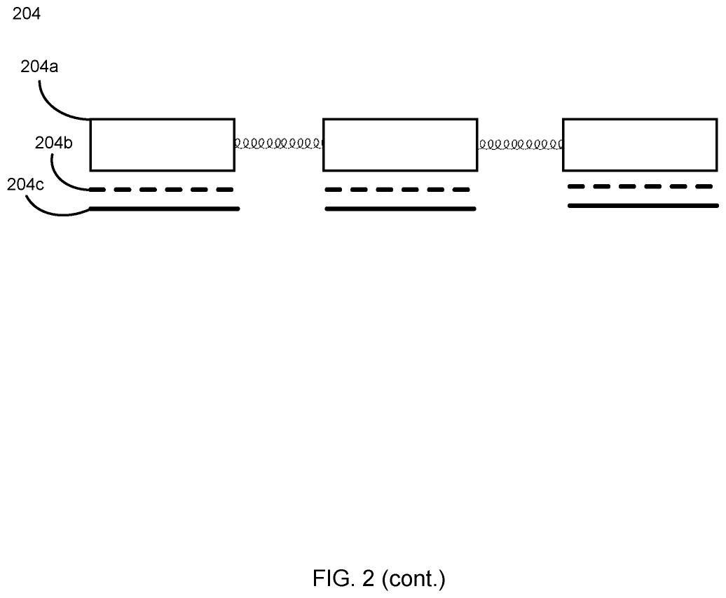

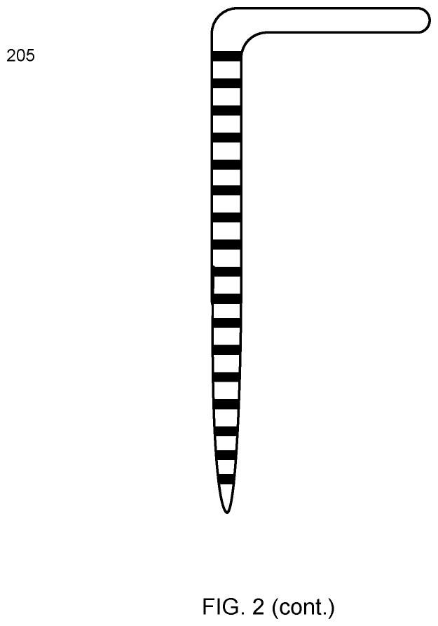

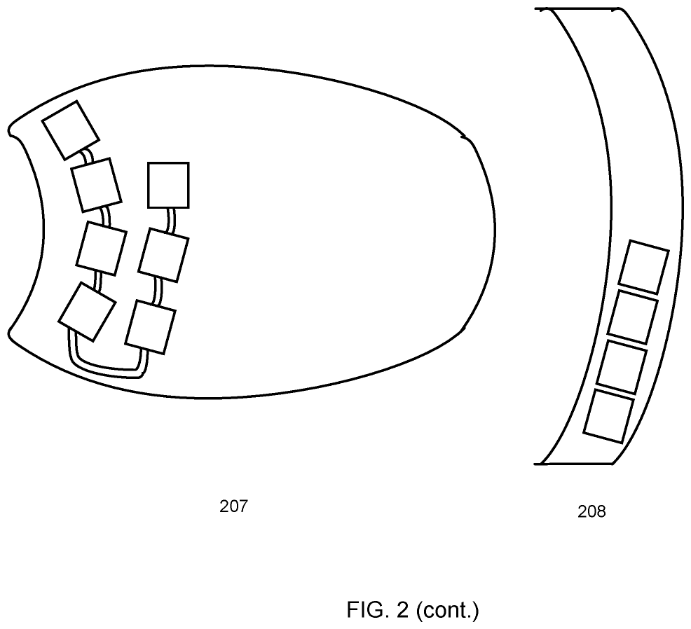

FIG. 2 illustrates a non-limiting example of a set of configurations of electrodes described herein. The electrodes can be integrated in a flexible circuit board or in a textile to be worn on a body part. One of the significant challenges in textiles is routing many and/or tightly spaced conductive threads. Using electrodes in a textile form allows many to be daisy chained with only a few conductive paths. The configurations can be used for electrocorticography (ECOG), electric muscular stimulation (EMS), or electroencephalography.

The electrodes can be electrically coupled to a stimulation module, using, for example, a wire, coiled wire, retractable wire, or within a textile or fabric. In some embodiments, a return electrode or ground electrode is needed to complete an electrode circuit via an additional wire and separate electrode. In some embodiments, a pair of electrodes comprises an integrated return and ground electrode as a frame around an active (e.g., stimulating or recording) electrode. The frame can be, for example, a circular (201), rectangular, or square frame. Electrodes can be arranged in an array. In some cases, the electrode array can be arranged in a serpentine flex circuit, for example, a winding path 203 comprising a sequence of electrodes. In some embodiments, electrodes are placed within a rigid board, in a puck/patch configuration (204b). In this configuration, one or more electrodes is disposed under a puck, which may be connected to another puck by a short, flexible connector (e.g., a coiled wire), to allow bending, flexing, or stretching for wearable applications. In some embodiments, the electrodes contact the skin via a layer of hydrogel.

The daisy-chaining can be used for implantable electrodes to solve wiring issues for stimulation or for biosignal or neural recordings. For example, a set of stereoelectroencephalogram (SEEG) or deep brain stimulation (DBS) electrodes 205 can also be wired in the daisy chain fashion. Additionally, daisy chain arrays can be used for electrocorticography (ECoG) arrays 207a or strips 207b.

### Machine Learning

An adaptive controller comprising a machine learning algorithm (e.g., a neural network) can learn to associate particular stimulation patterns with particular body movements. The controller can then drive the stimulation electrodes in spatiotemporal patterns to promote that a subject mimic hand or body movements (joint angles and force levels) performed by a therapist, caretaker, or user (able-bodied side).

The wearable system can use a classification model to classify body movements (e.g., exercises or portions thereof or functional movements or portions thereof). The classifier can be trained using data collected from the IMU of the wearable system to associate IMU measurements (e.g., accelerometer measurements, gyroscope measurements, and/or magnetometer measurements of translation (or displacement), rotation, velocity, acceleration, jerk, location, or other quantities) taken while the wearable device is worn with particular motions of the body part on which the wearable device is worn. The classifier can be trained on IMU measurements from a target subject or a population of subjects (whether or not similar to the target subject, where similarity can be based on demographic information, medical information, or similarity of injury, disease, or health condition).

In some embodiments, the classifier is a neural network. The neural network can comprise a long short-term memory (LSTM) network. The LSTM can be used to analyze motion of a subject that is undergoing rehabilitation or strength training. As a subject's condition improves, the LSTM can forget IMU measurements that were taken earlier during training and thus would not be relevant for training or fine-tuning the machine learning model. In this way, an LSTM produces accurate predictions of motions of the individual.

In some embodiments, the machine learning model predicts motion by processing patterns of electrical impulses (e.g., provided during neurostimulation or electrical muscle stimulation) and associating the patterns with motion data (e.g., from the IMU). A machine learning model is trained to predict a particular motion produced by a particular pattern of electrical impulses. Additionally, a machine learning model can be trained to predict a particular pattern of electrical impulses that produced an observed motion or set of motions. An adaptive controller can observe a set of motions performed by a therapist or other professional and generate a corresponding sequence of impulses so the subject mimics the therapist's motion.

Machine learning models can additionally be trained using both the observed motion data (e.g., from the IMU) and the corresponding electrical impulses. For example, if a motion produced by a subject from a set of electrical impulses deviates from that performed by a healthy individual, the machine learning model can predict a health condition, disease, disorder, or injury afflicting the subject, and prescribe a course of treatment. Machine learning models can be implemented while a subject is undergoing treatment to test the efficacy of a treatment. For example, IMU and electrical impulse data can be tested over many iterations during successive therapy appointments. The machine learning model can predict whether a therapy can be successful, how long the therapy can take, or be used to determine whether a new therapy is necessary.

In addition to analyzing IMU data and/or electrical impulse data, machine learning models described herein can analyze medical data (e.g., from an electronic health record (EHR)), demographic data, visual data (e.g., a video of exercise performance), or other data.

a. Training Phase

A machine learning software module as described herein can be configured to undergo at least one training phase wherein the machine learning software module is trained to carry out one or more tasks including data extraction, data analysis, and generation of output.

In some embodiments of the software application described herein, the software application comprises a training module that trains the machine learning software module. The training module is configured to provide training data to the machine learning software module, said training data comprising, for example, IMU measurements and the corresponding body motions associated with the IMU measurements (e.g., accelerometer measurements, gyroscope measurements, and/or magnetometer measurements of translation (or displacement), rotation, velocity, acceleration, jerk, location, or other quantities). In some embodiments, the training data comprise simulated IMU data with corresponding simulated body motions. In some embodiments, a machine learning software module utilizes automatic statistical analysis of data to determine which features to extract and/or analyze from an IMU measurement. In some embodiments, the machine learning software module determines which features to extract and/or analyze from an IMU measurement based on the training that the machine learning software module receives.

In some embodiments, a machine learning software module is trained using a data set and a target in a manner of supervised learning. In some embodiments, the data set is divided into a training set, a test set, and, in some embodiments, a validation set. A target is specified that contains the correct classification of each input value in the data set. For example, a set of IMU data from one or more individuals is repeatedly presented to the machine learning software module, and for each sample presented during training, the output generated by the machine learning software module is compared with the desired target. The difference between the target and the set of input samples is calculated, and the machine learning software module is modified to cause that the output more closely approximates the desired target value. In some embodiments, a back-propagation algorithm is utilized to cause that the output more closely approximates the desired target value. After a large number of training iterations, the machine learning software module output closely matches the desired target for each sample in the input training set. Subsequently, when new input data, not used during training, are presented to the machine learning software module, the module can generate an output classification value indicating into which of the categories the new sample is most likely to fall. The machine learning software module can generalize from the training to interpret new, previously unseen input samples. This feature of a machine learning software module allows classification of almost any input data that has a mathematically formulatable relationship to the category to which the data should be assigned.

In some embodiments, the machine learning software module utilizes an individual learning model. An individual learning model is based on the machine learning software module having trained on data from a single individual and thus, a machine learning software module that utilizes an individual learning model is configured to be used on a single individual on whose data the module was trained.

In some embodiments, the machine training software module utilizes a global training model. A global training model is based on the machine training software module having trained on data from multiple individuals and thus, a machine training software module that utilizes a global training model is configured to be used on multiple individuals.

In some embodiments, the machine training software module utilizes a simulated training model. A simulated training model is based on the machine training software module having trained on data from simulated IMU measurements.

In some embodiments, the use of training models changes as the availability of IMU data changes. For instance, a simulated training model can be used if insufficient quantities of appropriate motion data are available for training the machine training software module to a desired accuracy. This data scarcity can be true in the early days of implementation, if few appropriate IMU measurements with associated abnormalities are available initially. As additional data become available, the training model can change to a global or individual model. In some embodiments, a mixture of training models can be used to train the machine training software module. For example, a simulated and global training model can be used, utilizing a mixture of multiple subjects' data and simulated data to meet training data requirements.

Unsupervised learning is used, in some embodiments, to train a machine training software module to use input data such as, for example, IMU data and output, for example, a diagnosis or body motion. Unsupervised learning, in some embodiments, includes feature extraction, which is performed by the machine learning software module on the input data. Extracted features can be used for visualization, for classification, for subsequent supervised training, and more generally for representing the input for subsequent storage or analysis. In some embodiments, each training case consists of a plurality of IMU data.

Machine learning software modules that are suitable for unsupervised training include k-means clustering, mixtures of multinomial distributions, affinity propagation, discrete factor analysis, hidden Markov models, Boltzmann machines, restricted Boltzmann machines, autoencoders, convolutional autoencoders, recurrent neural network autoencoders, and long short-term memory autoencoders.

A machine learning software module can include a training phase and a prediction phase. The training phase provides data to train the machine learning algorithm. Non-limiting examples of types of data inputted into a machine learning software module for training include medical image data, clinical data (e.g., from a health record), encoded data, encoded features, and metrics derived from IMU data. Data that are inputted into the machine learning software module are used, in some embodiments, to construct a hypothesis function to determine the presence of a body motion. In some embodiments, a machine learning software module is configured to determine whether the outcome of the hypothesis function was achieved and based on that analysis to make a determination with respect to the data upon which the hypothesis function was constructed. That is, the outcome tends either to reinforce the hypothesis function with respect to the data upon which the hypothesis functions was constructed or to contradict the hypothesis function with respect to the data upon which the hypothesis function was constructed. In some embodiments, depending on how close the outcome tends to be to an outcome determined by the hypothesis function, the machine learning algorithm can adopt, adjust, or abandon the hypothesis function with respect to the data upon which the hypothesis function was constructed. As such, the machine learning algorithm described herein dynamically learns through the training phase what characteristics of an input (e.g., data) are most predictive in determining whether the features of a subject's recorded IMU data are predictive of a particular body motion.

For example, a machine learning software module is provided with data on which to train so that the module, for example, is able to determine the most salient features of a received IMU data to operate on. The machine learning software modules described herein can train on how to analyze the IMU data, rather than analyzing the IMU data using predefined instructions. As such, the machine learning software modules described herein dynamically learn through training what characteristics of an input measurement are most predictive in determining whether the features of an IMU display body motion.

In some embodiments, the machine learning software module is trained by repeatedly presenting the machine learning software module with IMU data along with, for example, body motions, for example, flexion, relaxation, stretching, or contraction of muscles. IMU data can comprise accelerometer, gyroscope, or magnetometer data produced responsive to electrical stimulation of a human body part.

In some embodiments, training begins when the machine learning software module is given IMU data and asked to determine the presence of a body motion. The predicted body motion is then compared to the true body motions that corresponds to the IMU data. An optimization technique such as gradient descent and backpropagation is used to update the weights in each layer of the machine learning software module to produce closer agreement between the body motion probability predicted by the machine learning software module, and the presence of the body motion. This process is repeated with new IMU data and body motions until the accuracy of the network has reached the desired level. In this case, training begins when the machine learning software module is given the corresponding IMU data and asked to determine the type and location of the body motion. An optimization technique is used to update the weights in each layer of the machine learning software module to produce closer agreement between the body motions predicted by the machine learning software module, and the true body motions. This process is repeated with new IMU data and body motions until the accuracy of the network has reached the desired level. The output data are then compared to the true body motions that corresponds to the IMU data. An optimization technique is used to update the weights in each layer of the machine learning software module to produce closer agreement between the body motion probability predicted by the machine learning software module, and the actual body motion. This process is repeated with new IMU data and body motions until the accuracy of the network has reached the desired level. Following training with the appropriate body motions given above, the machine learning module can analyze an IMU measurement and determine the presence of a body motion, the type and location of the body motion, and the conditions associated with such.

In some embodiments, the machine learning software module receives IMU data and directly determines the body motion probability of the subject, wherein the body motion probability comprises the probability that the IMU measurement is associated with the body motion of the subject.

In some embodiments, a user's body motions are inputted by the user of the system. In some embodiments, a user's body motions are inputted by an entity other than the user. In some embodiments, the entity can be a healthcare provider, healthcare professional, family member, or acquaintance. In some embodiments, the entity can be the instantly described system, device, or an additional system that analyzes IMU measurements and provides data pertaining to physiological abnormalities.

In some embodiments, a strategy for the collection of training data is provided to ensure that the IMU measurements represent a wide range of conditions to provide a broad training data set for the machine learning software module. For example, a prescribed number of measurements during a set period of time can be useful as a section of a training data set. Additionally, these measurements can be prescribed as having a set amount of time between measurements. In some embodiments, IMU measurements taken with variations in a subject's physical state can be included in the training data set.

b. Prediction Phase

Following training, the machine learning algorithm is used to determine, for example, the presence or absence of a body motion on which the system was trained using the prediction phase. With appropriate training data, the system can identify the type of a body motion, and present conditions associated with such body motion. For example, an IMU measurement is taken of a subject's brain and appropriate data derived from the IMU measurement is submitted for analysis to a system using the described trained machine learning algorithm. In some embodiments, a machine learning software algorithm detects a body motion associated with a health condition.

As an example, a subject is known to possess a health condition affecting mobility and has IMU measurements recorded before and after treatment with a therapy. The data from the IMU measurements and/or features and/or metrics derived from the said data are submitted for analysis to a system using the described trained machine learning algorithm to determine the effectiveness of the therapy using the prediction phase.

The prediction phase uses the constructed and optimized hypothesis function from the training phase to predict the probability of a body motion. In some embodiments, in the prediction phase, the machine learning software module can be used to analyze data derived from its IMU measurement independent of any system or device described herein. In some embodiments, the new data recording provides a longer signal window than that required for determining the presence of a subject's body motion. In some embodiments, the longer signal can be cut to an appropriate size, for example 10 seconds, and then can be used in the prediction phase to predict the probability of a body motion of the new patient data.

In some embodiments, a probability threshold can be used in conjunction with a final probability to determine whether a given recording matches the trained body motion. In some embodiments, the probability threshold is used to tune the sensitivity of the trained network. For example, the probability threshold can be 1%, 2%, 5%, 10%, 15%, 20%, 25%, 30%, 35%, 40%, 45%, 50%, 55%, 60%, 65%, 70%, 75%, 80%, 85%, 90%, 95%, 98%, or 99%. In some embodiments, the probability threshold is adjusted if the accuracy, sensitivity, or specificity falls below a predefined adjustment threshold. In some embodiments, the adjustment threshold is used to determine the parameters of the training period. For example, if the accuracy of the probability threshold falls below the adjustment threshold, the system can extend the training period and/or require additional measurements and/or body motions.

In some embodiments, additional measurements and/or body motions can be included into the training data. In some embodiments, additional measurements and/or body motions can be used to refine the training data set.

### Example Machine Learning Techniques

In some embodiments, the systems, methods, computer-readable media, and techniques disclosed herein use various machine learning techniques. In some embodiments, ML involves identifying and recognizing patterns in existing data to facilitate making predictions for subsequent data. ML can include a ML model for example, a ML algorithm. Machine learning, whether analytical or statistical, can provide deductive or abductive inference based on real or simulated data. The ML model can be a trained model. ML techniques can comprise one or more supervised, semi-supervised, self-supervised, or unsupervised ML techniques. For example, a ML model can be a trained model that is trained through supervised learning (e.g., various parameters are determined as weights or scaling factors). ML can comprise one or more of regression analysis, regularization, classification, dimensionality reduction, ensemble learning, meta learning, association rule learning, cluster analysis, anomaly detection, deep learning, or ultra-deep learning. Non-limiting examples of ML include: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation (LASSO), least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, generative adversarial networks, vision transformers, long short-term memory networks (LSTM), and masked autoencoders.

### Training

Training the ML model can include, in some embodiments, selecting one or more untrained data models to train using a training data set. The selected untrained data models can include any type of untrained ML models for supervised, semi-supervised, self-supervised, or unsupervised machine learning. The selected, untrained data models can be specified based on input (e.g., user input) specifying relevant parameters to use as predicted variables or other variables to use as potential explanatory variables. For example, the selected, untrained data models can be specified to generate an output (e.g., a prediction) based upon the input. Conditions for training the ML model from the selected untrained data models can likewise be selected, such as limits on the ML model complexity or limits on the ML model refinement past a certain point. The ML model can be trained (e.g., via a computer system such as a server) using the training data set. In some embodiments, a first subset of the training data set can be selected to train the ML model. The selected, untrained data models can then be trained on the first subset of training data set using appropriate ML techniques, based upon the type of ML model selected and any conditions specified for training the ML model. In some embodiments, due to the processing power requirements of training the ML model, the selected untrained data models are trained using additional computing resources (e.g., cloud computing resources). Such training can continue, in some embodiments, until at least one aspect of the ML model is validated and meets selection criteria to be used as a predictive model.

### Validation

In some embodiments, one or more aspects of the ML model can be validated using a second subset of the training data set (e.g., distinct from the first subset of the training data set) to determine accuracy and robustness of the ML model. Such validation can include applying the ML model to the second subset of the training data set to make predictions derived from the second subset of the training data. The ML model can then be evaluated to determine whether performance is sufficient based upon the derived predictions. The sufficiency criteria applied to the ML model can vary depending upon the size of the training data set available for training, the performance of previous iterations of trained models, or user-specified performance requirements. If the ML model does not achieve sufficient performance, additional training can be performed. Additional training can include refinement of the ML model or retraining on a different first subset of the training dataset, after which the new ML model can again be validated and assessed. When the ML model has achieved sufficient performance, in some embodiments, the ML can be stored for present or future use. The ML model can be stored as sets of parameter values or weights for analysis of further input (e.g., further relevant parameters to use as further predicted variables, further explanatory variables, further user interaction data, etc.), which can also include analysis logic or indications of model validity in some instances. In some embodiments, a plurality of ML models can be stored for generating predictions under different sets of input data conditions. In some embodiments, the ML model can be stored in a database (e.g., associated with a server).

### Deep Learning

The systems, methods, computer-readable media, and techniques disclosed herein can implement one or more deep-learning techniques. Deep learning is an example of ML that can be based on a set of algorithms that model high-level abstractions in data by using multiple processing layers, with complex structures or otherwise, composed of multiple non-linear transformations. In some embodiments, a drop out method can be used to reduce overfitting. At each training stage, individual nodes are either dropped out of the net (e.g., ignored) with probability 1-p or kept with probability p, so that a reduced network is left. Incoming and outgoing edges to a dropped-out node can also be removed. In some embodiments, the reduced network is trained on the data in that stage. The removed nodes can then be reinserted into the network with the original weights.

### Decision Tree

The systems, methods, computer-readable media, and techniques disclosed herein can implement one or more decision tree or random forest techniques. A decision tree can be a supervised ML algorithm that can be applied to both regression and classification problems. Decision trees can mimic the decision-making process of a human brain. For example, a decision tree can grow from a root (base condition), and when the tree meets a condition (internal node/feature), the tree splits into multiple branches. The end of the branch that does not split anymore is an outcome (leaf). A decision tree can be generated using a training data set according to the following operations: (1) Starting from a root node (the entire dataset), the algorithm can split the dataset in two branches using a decision rule or branching criterion; (2) each of the two branches can generate a new child node; (3) for each new child node, the branching process can be repeated until the dataset cannot be split any further; (4) each branching criterion can be chosen to maximize information gain (e.g., a quantification of how much a branching criterion reduces a quantification of how mixed the labels are in the children nodes). The labels can be the data or the classification that is predicted by the decision tree.

### Random Forest

A random forest regression is an extension of the decision tree model that tends to yield more robust predictions by stretching the use of the training data partition. Whereas a decision tree can make a single pass through the data, a random forest regression can bootstrap 50% of the data (e.g., with replacement) and build many trees. Rather than using all explanatory variables as candidates for splitting, a random subset of candidate variables can be used for splitting to produce trees that have different data and different variables. The predictions from the trees, collectively referred to as the forest, are then averaged to produce the final prediction. Many trees (e.g., one hundred trees) can be included in a random forest model, with a number (e.g., 3, 6, 10, etc.) of terms sampled per split, a minimum of number (e.g., 1, 2, 4, 10, etc.) of splits per tree, and a minimum split size (e.g., 16, 32, 64, 128, 256, etc.). Random forests can be trained in a similar way as decision trees. Training a random forest may include the following operations: (1) select randomly k features from the total number of features; (2) create a decision tree from these k features using the same operations as for generating a decision tree; and (3) repeat the previous two operations until a target number of trees is created.

### Long Short-Term Memory (LSTM)

The systems, methods, computer-readable media, and techniques disclosed herein can implement one or more long short-term memory (LSTM) techniques. LSTM can be an artificial neural network used in the fields of artificial intelligence and deep learning. Unlike standard feedforward neural networks, LSTM can use feedback connections. The LSTM architecture can provide a short-term memory for a recurrent neural network (RNN). Such RNN can process not only single data points (such as images), but also entire sequences of data (such as speech or video). The connection weights and biases in the RNN can change once per episode of training, analogously to how physiological changes in synaptic strengths store long-term memories. The activation patterns in the network can change once per time-step, analogously to how the moment-to-moment change in electric firing patterns in the brain store short-term memories. The LSTM architecture can provide a short-term memory for a RNN that can last many (e.g., thousands) timesteps.

In some embodiments, a LSTM unit can comprise a cell, an input gate, an output gate, and a forget gate. The cell can remember values over arbitrary time intervals and the input gate, the output gate, and the forget gate can regulate the flow of information into and out of the cell. Forget gates can be used to decide what information to discard from a previous state by assigning a previous state, compared to a current input, a value between 0 and 1 (e.g., a (rounded) value of 1 can mean to keep the information, and a value of 0 means to discard the information). The input gate can decide which pieces of new information to store in the current state, using the same system as the forget gates. The output gate can control which pieces of information in the current state to output (e.g., by assigning a value from 0 to 1 to the information, considering the previous and current states). Selectively outputting relevant information from the current state can allow that the LSTM network maintains useful, long-term dependencies to make predictions, both in current and future time-steps. LSTM networks can be well-suited to classifying, processing, and making predictions based on time series data, since lags of unknown duration between important events in a time series can occur. LSTMs can resolve the vanishing gradient problem that can be encountered when training traditional RNNs. Relative insensitivity to gap length can be an advantage of LSTM over RNNs, hidden Markov models, and other sequence learning methods in numerous applications.

In some embodiments, LSTMs can be used with one or more various types of neural networks (e.g., convolutional neural networks (CNNs), deep neural network (DNNs), recurrent neural networks (RNNs), etc.). In some embodiments, CNNs, LSTM, and DNNs are complementary in modeling capabilities and can be combined in a unified architecture. For example, in such unified architecture, CNNs can be well-suited at reducing frequency variations, LSTMs can be well-suited at temporal modeling, and DNNs can be well-suited for mapping features to a more separable space. For example, input features to a ML model using LSTM techniques in the unified architecture can include segment features for each of a plurality of segments. To process the input features for each of the plurality of segments, the segment features for the segment can be processed using one or more CNN layers to generate first features for the segment. The first features can be processed using one or more LSTM layers to generate second features for the segment. The second features can be processed using one or more fully connected neural network layers to generate third features for the segments, where the third features can be used for classification operations. In some embodiments, to process the first features using the one or more LSTM layers to generate the second features, the first features can be processed using a linear layer to generate reduced features having a reduced dimension from a dimension of the first features. The reduced features can be processed using the one or more LSTM layers to generate the second features. Short-term features having a first number of contextual frames can be generated based on the input features, where features generated using the one or more CNN layers can include long-term features having a second number of contextual frames that are more than the first number of contextual frames of the short-term features. In some embodiments, the one or more CNN layers, the one or more LSTM layers, and the one or more fully connected neural network layers can be jointly trained to determine trained values of parameters of the one or more CNN layers, the one or more LSTM layers, and the one or more fully connected neural network layers. In some embodiments, the input features include log-mel features having multiple dimensions. The input features include one or more contextual frames indicating a temporal context of a signal (e.g., input data). Implementations for such unified architecture can leverage complementary advantages associated with each of a CNN, LSTM, and DNN. For example, convolutional layers can reduce spectral variation in input and help the modeling of LSTM layers. Having DNN layers after LSTM layers can help reduce variation in the hidden states of the LSTM layers. Training the unified architecture jointly can provide a better overall performance. Training in the unified architecture can also remove the need to have separate CNN, LSTM and DNN architectures. By adding multi-scale information into the unified architecture, information can be captured at different time scales.

### Support Vector Machines

The systems, methods, computer-readable media, and techniques disclosed herein can implement one or more support vector machine learning techniques. In machine learning, support vector machines (SVMs) can be supervised learning models with associated learning algorithms that analyze data for classification and regression analysis. SVMs can be a robust prediction method, being based on statistical learning. SVMs can be well-suited for domains characterized by the existence of large amounts of data, noisy patterns, or the absence of general theories.

SVMs can map input vectors into high dimensional feature space through non-linear mapping function, chosen a priori. In this high dimensional feature space, an optimal separating hyperplane can be constructed. The optimal hyperplane can then be used to determine things such as class separations, regression fit, or accuracy in density estimation. More formally, a SVM constructs a hyperplane or set of hyperplanes in a high or infinite-dimensional space, which can be used for classification, regression, or other tasks like outlier detection.

Support vectors can be defined as the data points that lie closest to the decision surface (or hyperplane). Support vectors can therefore be the data points that are most difficult to classify and can have direct bearing on the optimum location of the decision surface. Given a set of training examples, each marked as belonging to one of two categories, a SVM training algorithm can build a model that assigns new examples to one category or the other, making the algorithm a non-probabilistic binary linear classifier. SVM can map training examples to points in space to maximize the width of the gap between the two categories. New examples can then be mapped into that same space and predicted to belong to a category based on which side of the gap the examples fall. In addition to performing linear classification, SVMs can efficiently perform a non-linear classification using what is called the kernel trick, implicitly mapping inputs into high-dimensional feature spaces.

Within a support vector machine, the dimensionally of the feature space can be large. For example, a fourth-degree polynomial mapping function can cause a 200-dimensional input space to be mapped into a 1.6 billionth dimensional feature space. SVMs assist in discovering knowledge from vast amounts of input data.

### Gradient Boosting

The systems, methods, computer-readable media, and techniques disclosed herein can implement one or more gradient boosting techniques. Gradient boosting is a machine learning technique used in regression and classification tasks, among others. Gradient boosting gives a prediction model in the form of an ensemble of weak prediction models, which are typically decision trees. When a decision tree is the weak learner, the resulting algorithm is called gradient-boosted trees. A gradient-boosted trees model is built in a stage-wise fashion as in other boosting methods, but generalizes the other methods by allowing optimization of an arbitrary differentiable loss function.

k-Nearest Neighbors

The systems, methods, computer-readable media, and techniques disclosed herein can implement one or more K-nearest neighbors (KNN) techniques. KNN is a non-parametric classification method. In KNN classification, the output is a class membership. An object is classified by a plurality vote of neighbors, with the object being assigned to the class most common among the k nearest neighbors (k is a positive integer, typically small). If k=1, then the object is assigned to the class of that single nearest neighbor. In KNN regression, the output is the property value for the object. This value is the average of the values of k nearest neighbors. KNN is a type of classification wherein the function is approximated locally and computation is deferred until function evaluation. Since this algorithm relies on distance for classification, if the features represent different physical units or come in vastly different scales, then normalizing the training data can improve accuracy.

The systems, methods, computer-readable media, and techniques disclosed herein can implement one or more Monte Carlo techniques. Monte Carlo is a broad class of computational algorithms that rely on repeated random sampling to obtain numerical results. The underlying concept is to use randomness to solve problems that might be deterministic in principle.

### One-Hot Encoding

The systems, methods, computer-readable media, and techniques disclosed herein can implement one or more one-hot encoding techniques. One-hot encoding can be used to deal with categorical data. For example, a ML model can use input variables that are numeric. The categorical variables can be transformed in a pre-processing part. Categorical data can be either nominal or ordinal. Ordinal data can have a ranked order of values and can therefore be converted to numerical data through ordinal encoding.

### Examples

The following examples describe the control of neurostimulation via a wearable system. The wearable system comprises a plurality of components as illustrated in FIG. 6, in particular, an extensor disposable electrode array, an extensor patch corresponding to the extensor disposable electrode array, a flexor disposable electrode array, a flexor patch corresponding to the flexor disposable electrode array, a stimulator, a thumb disposable electrode array, flexor/extensor cables, and a thumb dongle. The stimulator is paired with a user's personal device (e.g., mobile device). The user is allowed to use the personal device to control the neurostimulation of the wearable patch system wirelessly.

### Example 1: Attaching Disposable Electrode Array to Patient Skin

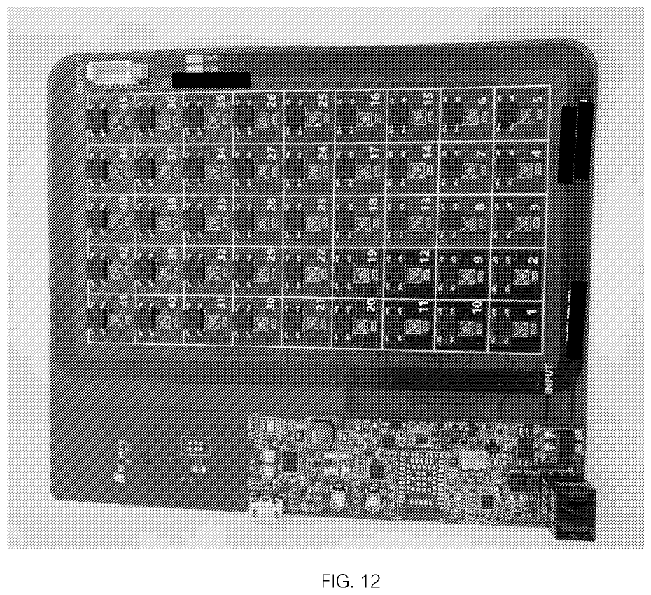

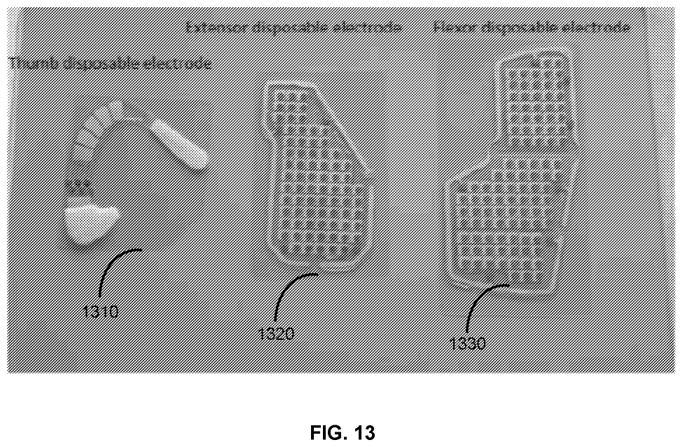

FIG. 13 illustrates a disposable electrode array configured to be attached to a patient's forearm and thumb. As illustrated, the disposable electrode array comprises a thumb disposable electrode array 1310, an extensor disposable electrode array 1320, and a flexor disposable electrode array 1330. The thumb disposable electrode array 1310 is configured to be worn around the thumb of the patient. The extensor disposable electrode array 1320 is configured to be attached to the extensor compartment (also called posterior compartment) of the forearm, stimulating the muscles (e.g., superficial muscles and deep muscles) and neural targets in the compartment. The flexor disposable electrode array 1330 is configured to be attached to the flexor compartment (also called anterior compartment) of the forearm, stimulating the muscles and neural targets in the compartment. As illustrated, both the extensor disposable electrode array 1320 and the flexor disposable electrode array 1330 comprise a plurality of electrodes, each electrode is attached to a corresponding magnetic connector. The disposable electrode arrays 1310, 1320, and 1330 are attached to a transparent sheet that can be removed from the electrodes when attached to the patient skin.

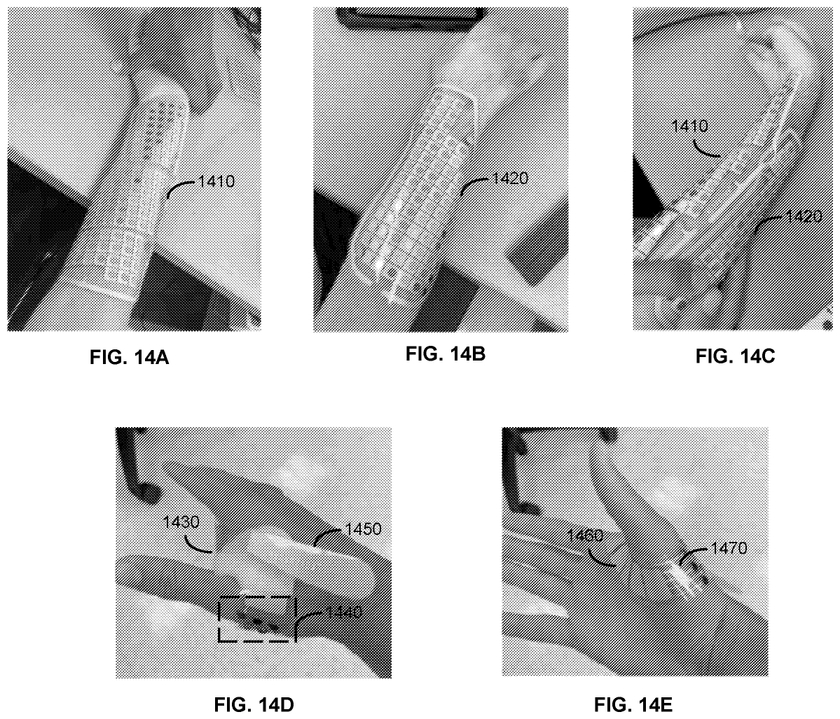

FIGS. 14A-14E illustrate a disposable electrode array that is attached to a patient's forearm and thumb. FIG. 14A illustrates a flexor disposable electrode array 1410 attached to the flexor compartment of a patient's forearm. FIG. 14B illustrates an extensor disposable electrode array 1420 attached to the extensor compartment of the patient's forearm. FIG. 14C is a side view of the patient's arm which the extensor disposable electrode array 1420 and flexor disposable electrode array 1410 are attached to and wrap around the arm. As shown in FIG. 14C, when the flexor and extensor disposable electrode array are attached, a substantial portion of the muscles and neural targets on the forearm are covered.

FIGS. 14D and 14E illustrate a disposable electrode array which is attached to and wraps around patient's thumb. The magnetic connectors 1440 are placed down on the ligament just beneath the bone connected to the thumb. The velcro area 1430 sits on the web of the hand, and the remaining flap 1460 wraps around the thumb. The thin end of the flap 1450 has velcro that attaches to the other velcro area 1430 on the web of the hand. Each of the electrodes in the disposable electrode array 1470 is in direct contact with the patient's skin.

### Example 2: Attaching Durable/Reusable Patches to Patient Skin

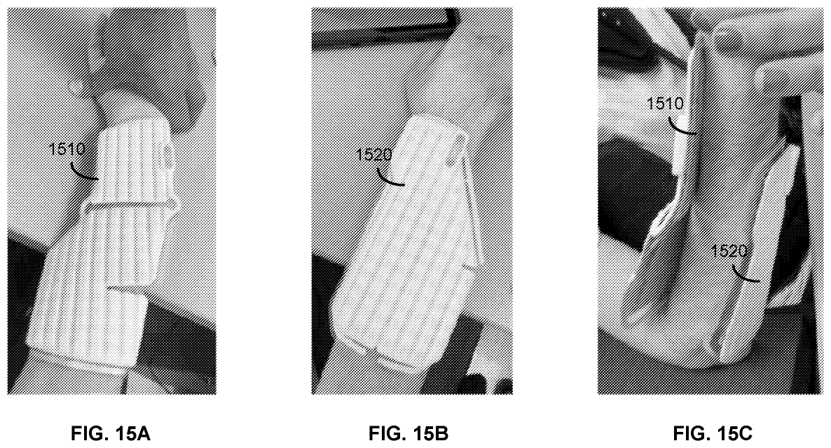

After the flexor and extensor disposable electrode arrays are attached to the patient's forearm, the corresponding durable/reusable electronic patches are placed above the disposable electrode arrays. FIG. 15A-15C show the durable/reusable electronic patches that are attached to the disposable electrode arrays. FIG. 15A illustrates a durable/reusable flexor patch 1510 corresponding to the flexor disposable electrode array 1410 attached to the patient's forearm, overlapping with the disposable electrode array. FIG. 15B illustrates a durable/reusable patch 1520 corresponding to the extensor disposable electrode array 1420 attached to the patient's forearm, overlapping the disposable electrode array. FIG. 15C is a side view of the patient's arm with the flexor patch 1510 and extensor patch 1520 attached thereto. The flexor patch 1510 has the same number of magnetic connectors as the magnetic connectors attached to the flexor disposable electrode array 1410, such that when the patch 1510 is placed above the electrode 1410, each magnetic connector in the patch can be aligned with the corresponding magnetic connector on the electrode. Similarly, the durable/reusable extensor patch 1520 has the same number of magnetic connectors as the magnetic connectors attached to the extensor disposable electrode array 1420. The thumb disposable electrode array does not require a patch to function.

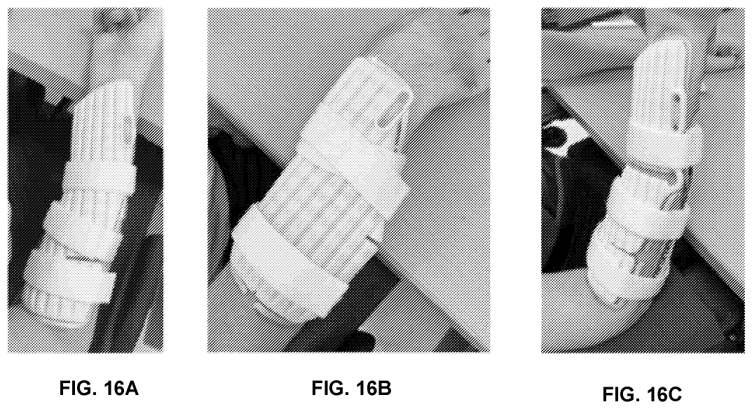

As further illustrated in FIG. 15C, when the durable/reusable patches are placed on the disposable electrode array, the electrodes sometimes slightly lift off the skin. To fix this lift, once the patch is placed on the disposable electrode array, the assembly can be secured. FIGS. 16A-16C illustrate the fixation of an assembly comprising a durable/reusable patch and corresponding disposable electrode array attached to the patient's forearm. A plurality of fixation mechanisms (e.g., straps) can be used, ensuring as much of the assembly touches the forearm as possible. If both the flexor and extensor patches are used at the same time, three forearm straps can be used, as illustrated in FIGS. 16A and 16C. If the extensor patch is used alone, two forearm straps can be used. The thumb electrode does not need this piece.

### Example 3. Establishing Connection Between Stimulator and Durable/Reusable Patch

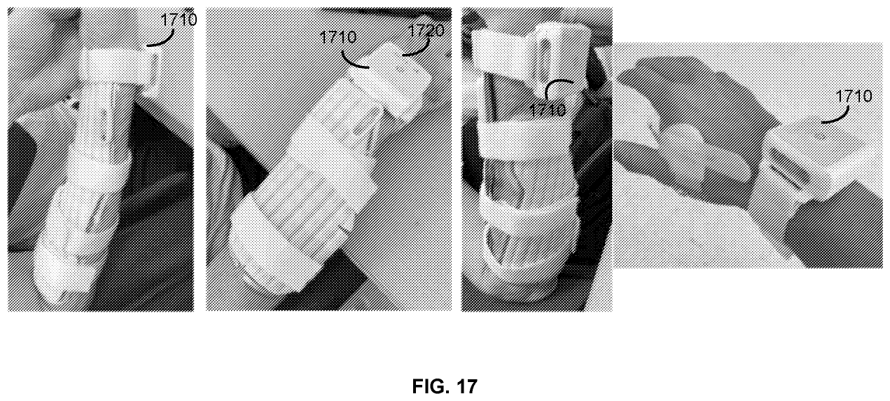

FIG. 17 illustrates a stimulator module attached to the wrist of the patient. The stimulator module 1710 is attached to a wrist strap and placed on the wrist of the patient. The LED sign 1720 on the stimulator module 1710 is closer to the fingers of the patient.

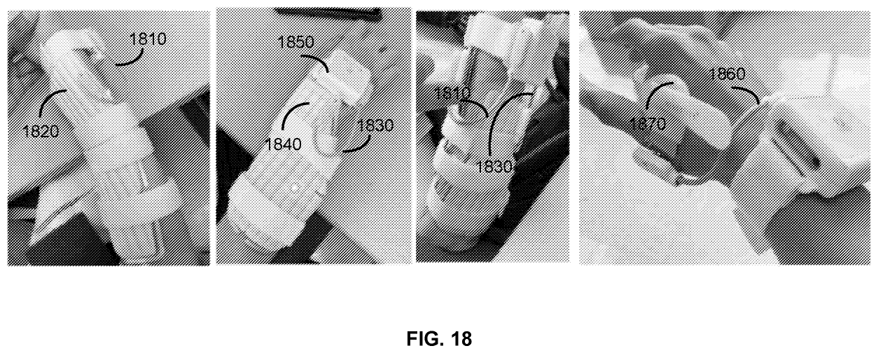

The stimulator module 1710 comprises three cable ports configured to be connected with the flexor and extensor patches as well as the thumb disposable electrode array. FIG. 18 illustrates the connections between the stimulator and the flexor and extensor patches and the thumb disposable electrode array via cables. A flexor cable 1810 is connected between the stimulator 1850 and the flexor patch 1820. An extensor cable 1830 is connected between the stimulator module 1850 and the extensor patch 1840. A thumb dongle 1860 is connected between the stimulator module 1850 and the thumb disposable electrode array. As such, the stimulator has established electrical connections with the flexor and extensor patches and the thumb disposable electrode array 1870.

### Example 4. Control of Neurostimulation via a Wearable Patch System

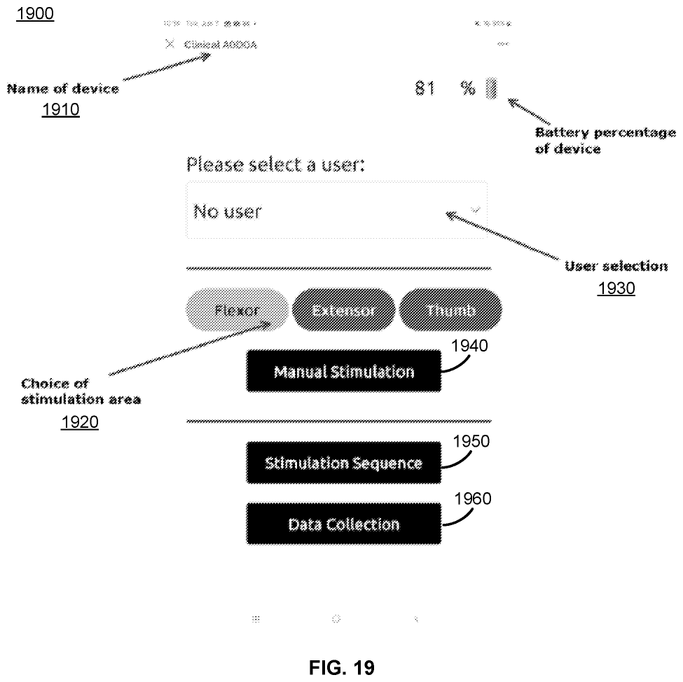

When the stimulator module is connected to the flexor and extensor patches and the thumb disposable electrode array, a user is allowed to operate on a personal device (e.g., mobile device) and control the patches and electrodes via a mobile application, a web application, or desktop application. FIG. 19 illustrates a graphical user interface (GUI) 1900 of an application that allows user control of the stimulation. The GUI lists the name of the device 1910, referring to the stimulator to which the patient's personal device is paired, and battery percentage of the device. The user is allowed to select the name of a patient on whom to perform stimulation. The application can store records of a plurality of patients. In a home care setting, the patient who wears the patch can operate the mobile device to control the stimulation. In a health care setting, a health care provider can operate on a web application or desktop application to select the name of the patient wearing the patch and control the stimulation.

The GUI also allows that a user select between manual stimulation 1940 and stimulation sequence 1950. In the manual stimulation section 1940, the user can select stimulation areas 1920, including flexor, extensor, and thumb. Each of these stimulation categories corresponds to the electrodes and patches, as illustrated in FIGS. 13-18. When a user selects a stimulation area, the user is allowed to control which electrodes perform stimulation. The user can save desired inputs for a specific motion during manual stimulation, including the positions for the stimulation and amplitude thereof. By selecting the stimulation sequence 1950, these saved motions can be performed sequentially or simultaneously.

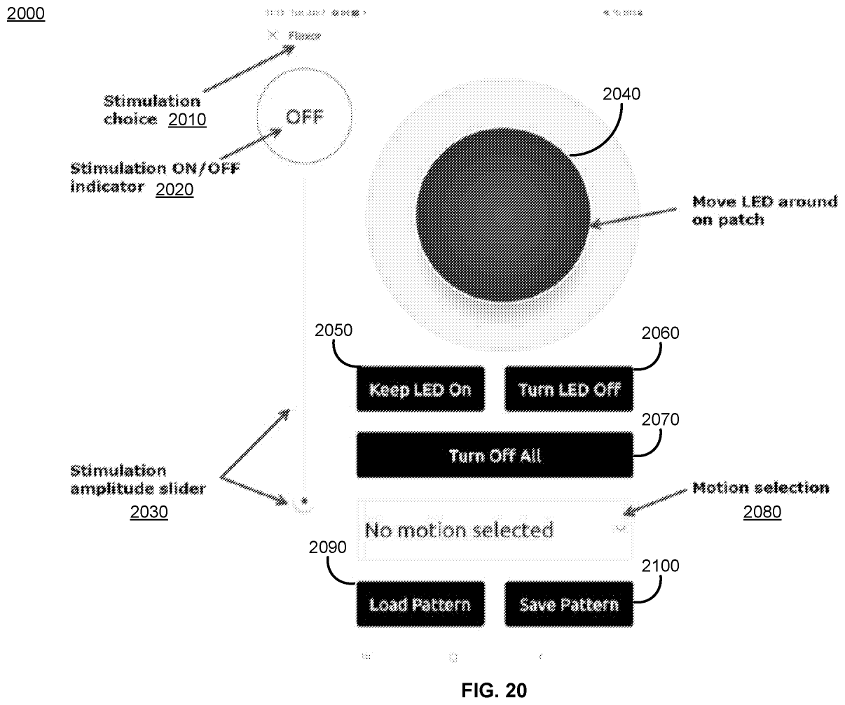

FIG. 20 illustrates a GUI 2000 of an application that allows that the user control manual stimulations. The user can select one of the flexor or extensor stimulation, where the result of the selection is shown in the stimulation choice section 2010. A stimulation indicator 2020 (e.g., “ON/OFF”) is also displayed in the GUI and automatically changes depending on the status of the stimulation amplitude slider 2030. For example, when the slider 2030 is dragged all the way down, indicating a minimum value of amplitude being applied, the stimulation indicator 2020 automatically changes to OFF. When the slider is dragged from the minimum upwards, the button automatically changes to ON. Alternatively, the stimulation indicator 2020 can be turned on and off independent from the amplitude slider 2030.

As described above in accordance with FIG. 18, the flexor and extensor patches have LEDs indicating the position of the electrodes that generate electrical stimulation. The user can control the position of the electrodes by navigating the controlling area 2040. A user can move the fingers on the controlling area 2040 to locate a desired spot to perform the stimulation. The LEDs on the patch corresponding to the desired spot can emit light, indicating the location of the electrodes that perform the stimulation. The user can also test different positions of the LEDs on the patch and see where the patient responds to the stimulation by navigating the controlling area 2040.

The GUI also provides Keep LED On function 2050. When the user identifies a desired spot for electrical stimulation indicated by the LEDs on the patch, the user can save the position of the LEDs by tapping the Keep LED On 2050. The selected LEDs, corresponding to the position of the desired spot for stimulation, can change to a different color. The user can keep navigating on the controlling area 2040 to identify a different desired spot for electrical stimulation. Another LED on the patch, representing the spot that the user newly selects, can emit light.

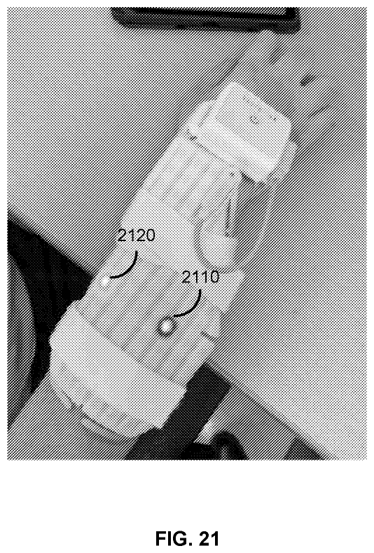

FIG. 21 illustrates user's selection of multiple positions on the patch to perform electrical stimulation, each position being indicated by a LED. The position of blue LED 2110 represents a first desired spot that the user selected and saved by tapping the “Keep LED On” button 2050. The position of green LED 2120 represents a new position that the user is navigating on the controlling area 2040. When the user's finger slides on the controlling area 2040, the position of the green LED 2120 changes accordingly. When the user identifies a second desired spot for stimulation, the user taps the Keep LED On 2050 again to save this spot. Accordingly, the green LED 2120 turns blue, indicating the corresponding spot is selected and saved.

The GUI allows that the user save a pattern with one or more selected positions. As illustrated in FIG. 20, the user can tap Save Pattern 2100. The desired pattern can be saved in the user's profile. The user can load this pattern even after adjusting the inputs. In some embodiments, only one pattern can be saved at once for each selected motion. If another pattern is saved over an already saved pattern, the newly saved pattern can overwrite on the selected motion. When the user intends to remove a previously selected position represented by blue LED on the patch, the user can either tap Turn LED Off 2060, or tap Turn Off All 2070 to remove all of the selected positions. As each pattern is already saved in the user's profile, even after turning the blue LEDs off, the user can still tap Load Pattern 2090 to restore the previously saved patterns.

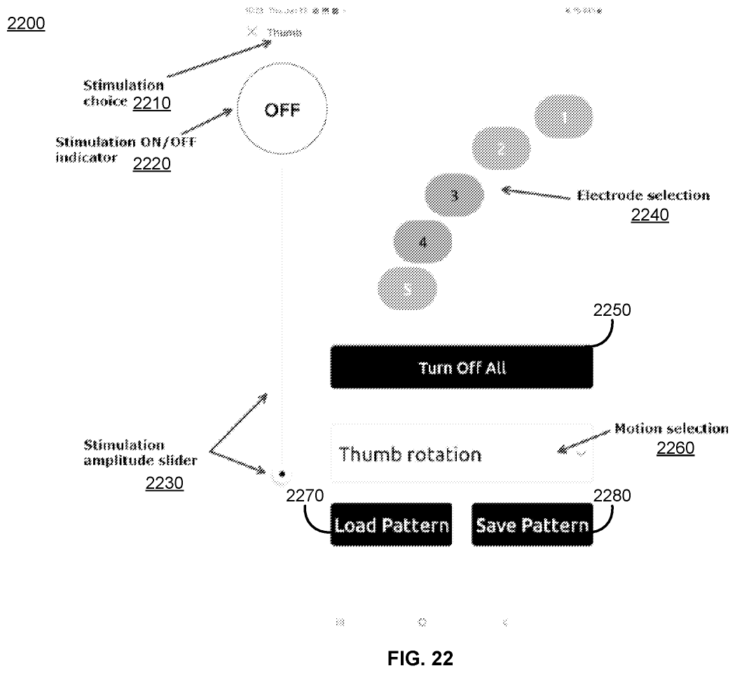

FIG. 22 illustrates a GUI 2200 of an application that allows that the user control thumb stimulations. Instead of selecting one of extensor or flexor stimulations, the user can select thumb stimulation, which is indicated in the stimulation choice section 2210. A stimulation indicator 2220 (e.g., ON/OFF) is also displayed in the GUI and automatically changes depending on the status of the stimulation amplitude slider 2230. For example, when the slider 2230 is dragged all the way down to indicate a minimum value of amplitude being applied, the stimulation indicator 2220 automatically changes to OFF. When the slider is dragged from the minimum upwards, the button automatically changes to ON. Alternatively, the stimulation indicator 2220 can be turned on and off independent from the amplitude slider 2230.

The GUI 2200 provides an electrode section 2240 comprising visual representations of five subsystem electrodes in the disposable thumb electrode (see five subsystem electrodes 1470 in FIG. 14E). Each of the electrodes can be selected to control different sections of the thumb movement. The user can select a single electrode or multiple electrodes at once, depending on the motion that the user selects in the motion selection section 2260. As illustrated, for example, electrodes 2 and 3 are selected simultaneously and generate electrical stimulations. The user can also tap Turn Off All 2250 to remove the selected positions.

The GUI 2200 also provides Load Pattern 2270 and Save Pattern 2280, which have similar functions to those in the flexor and extensor modes (e.g., 2090 and 2100 in FIG. 20). The user can save a desired pattern with a given amplitude and stimulation area by tapping the Save Pattern 2280. The saved pattern can be loaded by tapping the “Load Pattern” 2270.

As described above, the user can save desired inputs for a specific motion during manual stimulation, including the positions for the stimulation and amplitude thereof. The system allows that the user select Stimulation Sequence function such that the saved motions can be performed sequentially or simultaneously.

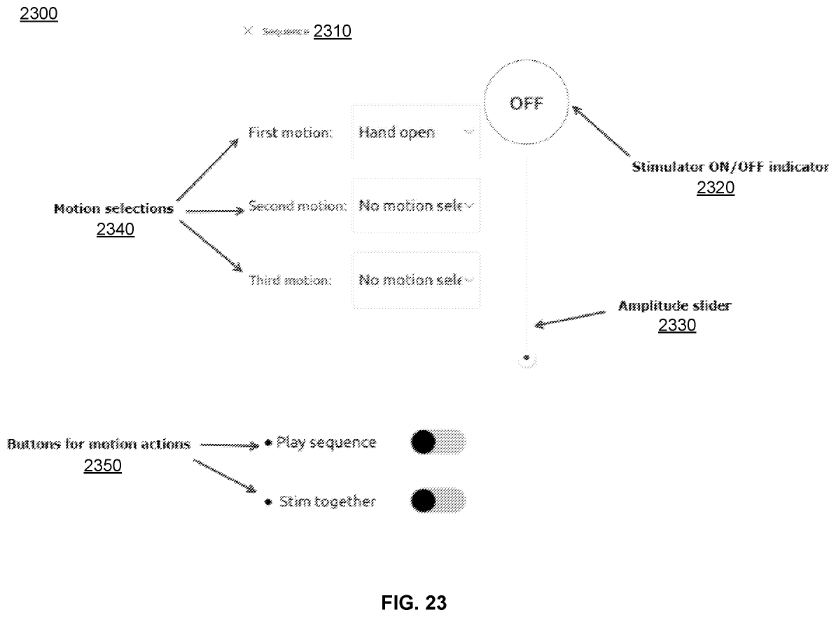

FIG. 23 illustrates a GUI 2300 of an application for performing stimulation sequence 2300. Instead of selecting manual stimulation, the user can select stimulation sequence 2310 where one or more saved motions can be performed. A stimulation indicator 2320 (e.g., ON/OFF) is displayed in the GUI and automatically changes depending on the status of the stimulation amplitude slider 2330. The functions of the stimulation indicator 2320 and stimulation amplitude slider 2330 are like those as described in FIGS. 20 and 22. The motion selection section 2340 comprises three saved motions to be selected by the user. As illustrated, for example, a Hand Open motion is saved by the user during manual stimulation. Similarly, the user can navigate the scroll-down menu to select any of the saved motions. The user can also select how the motions are performed in the motion action section 2350, for example, to be played in sequence or stim together.

### Embodiments

Embodiment 1. A device comprising: a first article, wherein the first article comprises 1) a first top surface, wherein the first top surface comprises a set of first connectors; and 2) a first bottom surface, wherein the first bottom surface comprises a plurality of electrodes; and a second article, wherein the second article comprises 1) a circuit board operably connected to the plurality of electrodes; 2) a second top surface, wherein the second top surface comprises a plurality of visualization aids, wherein each visualization aid of the plurality of visualization aids independently corresponds to one of the plurality of electrodes; and 3) a second bottom surface, wherein the second bottom surface comprises a set of second connectors, wherein each of the first connectors is independently configured to couple to one of the second connectors, wherein the first connectors and the second connectors are configured to form connections that hold the first article and the second article together when the first connectors are coupled to the second connectors, wherein when the first article and the second article are operably connected, the first article and the second article together form a wearable, wherein the wearable has a wearable size and a wearable shape adapted to fit on a human body part.

Embodiment 2. The device of embodiment 1, wherein the visualization aids are lights.

Embodiment 3. The device of embodiment 1 or 2, wherein the visualization aids are LEDs.

Embodiment 4. The device of any one of embodiments 1-3, wherein each visualization aid is configured to provide a visible signal when an electrode corresponding to the visualization aid emits an electrical signal.

Embodiment 5. The device of any one of embodiment 1-4, wherein the plurality of electrodes are flexible.

Embodiment 6. The device of any one of embodiments 1-5, wherein the plurality of electrodes are silver ink.

Embodiment 7. The device of any one of embodiments 1-6, wherein the plurality of electrodes are configured to stimulate muscle tissue and neural targets when the plurality of electrodes provide electrical stimulation to the muscle tissue and the neural targets.

Embodiment 8. The device of any one of embodiments 1-7, wherein the plurality of electrodes is connected in sequence.

Embodiment 9. The device of any one of embodiments 1-8, wherein the first connectors and the second connectors are magnetic.

Embodiment 10. The device of any one of embodiments 1-9, wherein the first article has a first article shape, and the second article has a second article shape, wherein when the first article and the second article are operably connected, the first article shape and the second article shape are substantially overlapping.

Embodiment 11. The device of any one of embodiments 1-10, wherein the first bottom surface has a surface area of at least 25 cm².

Embodiment 12. The device of any one of embodiments 1-11, wherein the circuit board is flexible.

Embodiment 13. The device of any one of embodiments 1-12, wherein the circuit board comprises a conductor.

Embodiment 14. The device of embodiment 13, wherein the conductor is gold-plated copper.

Embodiment 15. The device of embodiment 13 or 14, wherein the conductor is bonded to plating.

Embodiment 16. The device of any one of embodiments 13-15, wherein the conductor is bonded to nickel-copper-nickel plating.

Embodiment 17. The device of any one of embodiments 1-16, wherein the second article is durable.

Embodiment 18. The device of any one of embodiments 1-17, wherein the first article is disposable.

Embodiment 19. The device of any one of embodiments 1-18, wherein the first article is configured for contact with human skin.

Embodiment 20. The device of any one of embodiments 1-19, wherein the first article comprises a bottom layer, a middle layer, and a top layer, wherein the middle layer is layered on top of the bottom layer, is in contact with the bottom layer, and is operably connected to the bottom layer, and wherein the top layer is layered on top of the middle layer, is in contact with the middle layer, and is operably connected to the middle layer.

Embodiment 21. The device of embodiment 20, wherein the bottom layer comprises the plurality of electrodes.

Embodiment 22. The device of embodiment 20 or 21, wherein the bottom layer comprises a hydrogel and the plurality of electrodes.

Embodiment 23. The device of any one of embodiments 20-22, wherein the middle layer comprises a dielectric material.

Embodiment 24. The device of any one of embodiments 20-23, wherein the top layer comprises the set of first connectors.

Embodiment 25. The device of any one of embodiments 20-24, wherein the bottom layer has a bottom layer shape, the middle layer has a middle layer shape, and the top layer has a top layer shape, wherein when the bottom layer and the middle layer are operably connected, and the middle layer and the top layer are operably connected, then the bottom layer shape, the middle layer shape, and the top layer shape are substantially overlapping.

Embodiment 26. The device of any one of embodiments 1-25, further comprising c) a control system operably connected to the second article, wherein the control system comprises 1) a power supply; 2) a processor operably connected to the power supply and configured to operate the device; and 3) a stimulator operably connected to the processor, operably connected to the power supply, and configured to send electrical stimulation to the circuit board.

Embodiment 27. The device of embodiment 26, wherein the power supply, the processor, and the stimulator are in a common housing.

Embodiment 28. The device of embodiment 26 or 27, wherein the control system further comprises a wireless receiver operably connected to the processor and configured to receive instructions from a user for sending electrical stimulation to the circuit board.

Embodiment 29. The device of any one of embodiments 26-28, wherein the control system further comprises a memory system operably connected to the processor and configured to record information regarding use of the device.

Embodiment 30. The device of any one of embodiments 26-29, wherein the control system further comprises a transmitter operably connected to the processor and configured to transmit wirelessly to a recipient external to the device information regarding use of the device.

Embodiment 31. The device of embodiment 30, wherein the information regarding use of the device is a record of stimulation provided to a human body part in physical contact with the device and motion of the human body part in response to the stimulation.

Embodiment 32. The device of any one of embodiments 26-31, wherein the control system further comprises a motion detector operably connected to the processor and configured to detect motion of a human body part in physical contact with the device.

Embodiment 33. A device comprising: a) a first article, wherein the first article is disposable, wherein the first article comprises: 1) a bottom layer, wherein the bottom layer comprises: A) a hydrogel; and B) a plurality of electrodes in contact with the hydrogel, wherein the plurality of electrodes is connected in sequence, wherein the plurality of electrodes is flexible, wherein the plurality of electrodes are silver ink, wherein the plurality of electrodes are configured to stimulate muscle tissue and neural targets when the plurality of electrodes provide electrical stimulation to the muscle tissue and the neural targets; 2) a middle layer, wherein the middle layer comprises a dielectric material, wherein the middle layer is layered on top of the bottom layer, is in contact with the bottom layer, and is operably connected to the bottom layer; and 3) a top layer, wherein the top layer comprises a set of first connectors, wherein the first connectors are magnetic, wherein the top layer is layered on top of the middle layer, is in contact with the middle layer, and is operably connected to the middle layer, wherein the bottom layer has a bottom layer shape, the middle layer has a middle layer shape, and the top layer has a top layer shape, wherein when the bottom layer and the middle layer are operably connected, and the middle layer and the top layer are operably connected, then the bottom layer shape, the middle layer shape, and the top layer shape are substantially overlapping; b) a second article, wherein the second article is durable, wherein the second article comprises: 1) a second top surface, wherein the second top surface comprises a plurality of visualization aids, wherein each visualization aid of the plurality of visualization aids independently corresponds to one of the plurality of electrodes, wherein the plurality of visualizations aids are LEDs, wherein each visualization aid is configured to provide a visible signal when an electrode corresponding to a visualization aid emits an electrical signal; 2) a flexible circuit board comprising a conductor, the flexible circuit board being operably connected to the plurality of electrodes; and 3) a second bottom surface, wherein the second bottom surface comprises a set of second connectors, wherein the second connectors are magnetic, wherein each of the first connectors is independently configured to couple to one of the second connectors; and c) a control system operably connected to the second article, wherein the control system comprises: 1) a power supply; 2) a processor operably connected to the power supply and configured to operate the device; 3) a stimulator operably connected to the processor, operably connected to the power supply, and configured to send electrical stimulation to the flexible circuit board; 4) a wireless receiver operably connected to the processor and configured to receive instructions from a user for sending electrical stimulation to the flexible circuit board; 5) a memory system operably connected to the processor and configured to record information regarding use of the device; 6) a transmitter operably connected to the processor and configured to transmit wirelessly to a recipient external to the device a record of stimulation provided to a human body part in physical contact with the device and motion of the human body part in response to the stimulation; and 7) a motion detector operably connected to the processor and configured to detect motion of a human body part in physical contact with the device, wherein the power supply, the processor, the stimulator, the wireless receiver, the memory system, the transmitter, and the motion detector are in a common housing, wherein the first connectors and the second connectors are configured to form connections that hold the first article and the second article together when the first connectors are coupled to the second connectors, wherein when the first article and the second article are operably connected, the first article and the second article together form a wearable, wherein the wearable has a wearable size and a wearable shape adapted to fit on the human body part, wherein the first article has a first article shape, and the second article has a second article shape, wherein when the first article and the second article are operably connected, the first article shape and the second article shape are substantially overlapping, wherein the bottom layer has a surface area of at least 25 cm².

Embodiment 34. A system comprising a device of any one of embodiments 1-33 and a telecommunications instrument that is configured to operate the device wirelessly.

Embodiment 35. The system of embodiment 34, wherein the telecommunications device comprises non-transitory, computer-executable code encoded on a computer-readable medium, wherein the non-transitory, computer-executable code is configured to operate the device based on instructions provided by a user.

Embodiment 36. The system of embodiment 35, wherein the non-transitory, computer-executable code is configured to monitor performance of a human subject using the device in physical therapy.

Embodiment 37. The system of embodiment 35 or 36, wherein the non-transitory, computer-executable code is configured to provide analysis of a performance of a human subject using the device in therapy and to determine a proposed therapy regimen based on the analysis, wherein the proposed therapy regimen comprises activation of the plurality of electrodes in a pattern that is determined to be likely to provide a physical therapy improvement based on the analysis.

Embodiment 38. The system of any one of embodiments 35-37, wherein the telecommunications instrument comprises a touch screen that displays a grid, wherein the grid corresponds to a layout of the plurality of electrodes.

Embodiment 39. The system of any one of embodiments 35-38, wherein the telecommunications instrument comprises a touch screen that displays a grid, wherein the grid corresponds to a layout of the plurality of electrodes, wherein touching the touchscreen on a point on the grid activates an electrode of the plurality of electrodes that corresponds to the point on the grid.

Embodiment 40. A method comprising contacting to a human subject the device of any one of embodiments 1-33.

Embodiment 41. A method comprising: a) contacting a body part of a human subject with a plurality of electrodes, wherein the plurality of electrodes is operably connected to a flexible circuit board, wherein the body part has a shape; b) manipulating the flexible circuit board to conform substantially to the shape of the body part; c) operably connecting to the flexible circuit board a plurality of visualization aids, wherein each visualization aid of the plurality of visualization aids independently corresponds to one of the plurality of electrodes; and d) applying to the plurality of electrodes an electric current, whereupon the plurality of electrodes provide electrical stimulation to the body part.

Embodiment 42. The method of embodiment 41, wherein the body part comprises muscle tissue and neural targets.

Embodiment 43. The method of embodiment 41 or 42, wherein the plurality of electrodes are disposed in a hydrogel and the hydrogel is in contact with the body part.

Embodiment 44. The method of any one of embodiments 41-43, wherein the plurality of electrodes are silver ink.

Embodiment 45. The method of any one of embodiments 41-44, wherein each visualization aid is configured to provide a visible signal when an electrode corresponding to a visualization aid emits an electrical signal.

Embodiment 46. The method of embodiment 45, wherein the plurality of visualizations aids are LEDs.

Embodiment 47. The method of any one of embodiments 41-46, further comprising operably connecting a source of electrical current to the flexible circuit board.

Embodiment 48. The method of any one of embodiments 41-47, wherein the flexible circuit board is operably connected to a source of electric current.

Embodiment 49. The method of any one of embodiments 41-48, further comprising tracking motion of the body part in response to the electrical stimulation.

Embodiment 50. The method of any one of embodiments 41-49, further comprising tracking motion of the body part in response to the electrical stimulation and designing a physical therapy regimen based at least in part on the motion of the body part in response to the electrical stimulation, wherein the physical therapy regimen comprises a pattern of electrical signals applied via the plurality of electrodes.

Embodiment 51. A method comprising: a) contacting a body part of a human subject with a plurality of electrodes, wherein the plurality of electrodes is operably connected to a flexible circuit board, wherein the body part has a shape; b) manipulating the flexible circuit board to conform substantially to the shape of the body part; c) receiving instructions from a wireless user device for sending electrical stimulation to the flexible circuit board; and d) selecting at least a portion of the plurality of electrodes and applying to the portion of the plurality of electrodes an electric current, whereupon the portion of the plurality of electrodes provides the electrical stimulation to the body part.

Embodiment 52. The method of embodiment 51, wherein the body part comprises muscle tissue and neural targets.

Embodiment 53. The method of embodiment 51 or 52, wherein the plurality of electrodes are disposed in a hydrogel and the hydrogel is in contact with the body part.

Embodiment 54. The method of any one of embodiments 51-53, wherein the plurality of electrodes are silver ink.

Embodiment 55. The method of any one of embodiments 51-54, further comprising operably connecting to the flexible circuit board a plurality of visualization aids, wherein each visualization aid of the plurality of visualization aids independently corresponds to one of the plurality of electrodes, wherein each visualization aid is configured to provide a visible signal when an electrode corresponding to a visualization aid emits an electrical signal.

Embodiment 56. The method of any one of embodiments 51-55, wherein the flexible circuit board is operably connected to a plurality of visualization aids, wherein each visualization aid of the plurality of visualization aids independently corresponds to one of the plurality of electrodes, wherein each visualization aid is configured to provide a visible signal when an electrode corresponding to a visualization aid emits an electrical signal.

Embodiment 57. The method of embodiment 56, wherein the plurality of visualizations aids are LEDs.

Embodiment 58. The method of any one of embodiments 51-57, further comprising operably connecting a source of electrical current to the flexible circuit board.

Embodiment 59. The method of any one of embodiments 51-58, wherein the flexible circuit board is operably connected to a source of electric current.

Embodiment 60. The method of any one of embodiments 51-59, further comprising tracking motion of the body part in response to the electrical stimulation.

Embodiment 61. The method of any one of embodiments 51-60, further comprising tracking motion of the body part in response to the electrical stimulation and designing a physical therapy regimen based at least in part on the motion of the body part in response to the electrical stimulation, wherein the physical therapy regimen comprises a pattern of electrical signals applied via the plurality of electrodes.

Embodiment 62. An addressable electrode system, comprising: a power supply; and an article comprising: (i) a plurality of sequentially-connected electrodes; (ii) a plurality of indicators; and (iii) a plurality of switches, each switch independently comprising a first terminal, a second terminal, and a third terminal; wherein the first terminal of the switch is electrically coupled to the power supply; wherein the second terminal of the switch is electrically coupled to an indicator of the plurality of indicators; wherein the third terminal of the switch is electrically coupled to an electrode of the plurality of sequentially-connected electrodes; and wherein the switch is configured, when power is provided by the power supply to the first terminal of the switch, to provide electrical power simultaneously to the electrode and to the indicator.

Embodiment 63. The system of embodiment 62, wherein the article is a textile.

Embodiment 64. The system of embodiment 62 or 63, wherein article is subdivided into a plurality of patches.

Embodiment 65. The system of embodiment 64, wherein each indicator of the plurality of indicators independently corresponds to a patch of the plurality of patches.

Embodiment 66. The system of embodiment 65, wherein the indicator is disposed above the patch.

Embodiment 67. The system of any one of embodiments 62-66, wherein the plurality of sequentially-connected electrodes is connected in a daisy-chain configuration.

Embodiment 68. The system of any one of embodiments 62-67, wherein the switch comprises a solid-state relay.

Embodiment 69. The system of any one of embodiments 62-67, wherein the switch comprises a metal-oxide-semiconductor field effect transistor (MOSFET).

Embodiment 70. The system of any one of embodiments 62-69, wherein an indicator of the plurality of indicators and a switch of the plurality of switches are incorporated into a single integrated circuit (IC).

Embodiment 71. The system of any one of embodiments 62-70, wherein the second terminal of each switch is electrically coupled to at most one indicator.

Embodiment 72. The system of any one of embodiments 62-71, wherein the second terminal of each switch is electrically coupled to at most one electrode.

Embodiment 73. The system of any one of embodiments 62-72, wherein each electrode of the plurality of sequentially-connected electrodes is electrically coupled to a return electrode.

Embodiment 74. The system of embodiment 73, wherein each return electrode is integrated with one of the electrodes of the plurality of sequentially-connected electrodes.

Embodiment 75. The system of any one of embodiments 62-74, wherein the plurality of sequentially-connected electrodes comprises at least a first electrode and a second electrode, wherein the first electrode is connected to the second electrode with a coiled wire, wherein the coiled wire is flexible.

Embodiment 76. The system of any one of embodiments 62-75, wherein each indicator produces visible light.

Embodiment 77. The system of embodiment 76, wherein each indicator is a light-emitting diode (LED).

Embodiment 78. A method for providing neurostimulation, comprising: a) receiving at a user interface (UI) a gesture of a user, wherein the gesture is physical contact with the user interface; b) generating a control signal based at least in part on the gesture; c) generating, from the control signal, a spatial pattern of recommended neurostimulation impulses for a subject; and d) providing electrical current to at least one of a plurality of electrodes in contact with the subject to provide to the subject the spatial pattern of neurostimulation impulses.

Embodiment 79. The method of embodiment 78, wherein the user interface comprises a graphical user interface (GUI).

Embodiment 80. The method of embodiment 79, wherein the GUI comprises a layout, wherein the layout corresponds to a spatial configuration of the plurality of electrodes.

Embodiment 81. The method of embodiment 80, wherein the layout comprises at least two dimensions.

Embodiment 82. The method of embodiment 81, wherein the layout is a grid.

Embodiment 83. The method of any one of embodiments 78-81, further comprising displaying the user interface on a telecommunications device.

Embodiment 84. The method of embodiment 83, wherein the telecommunications device is a mobile device.

Embodiment 85. The method of any one of embodiments 78-84, wherein the user interface is provided by a software application.

Embodiment 86. The method of embodiment 85, wherein the software application is a mobile application.

Embodiment 87. The method of any one of embodiments 78-86, wherein the physical contact with the user interface occurs at a portion of the user interface, wherein the portion of the user interface corresponds to a location on the subject of at least one electrode of electrode of the plurality of electrodes.

Embodiment 88. The method of embodiment 87, wherein the physical contact originates from a hand or stylus.

Embodiment 89. The method of embodiment 88, wherein the physical contact comprises one or more taps.

Embodiment 90. The method of embodiment 89, wherein the one or more taps are simultaneous.

Embodiment 91. The method of embodiment 89, wherein the one or more taps are sequential in time.

Embodiment 92. The method of embodiment 87, wherein the physical contact comprises pressing for an extended period.

Embodiment 93. The method of embodiment 92, wherein the physical contact further comprises dragging.

Embodiment 94. The method of any one of embodiments 78-93, wherein the control signal is provided over a network.

Embodiment 95. The method of embodiment 94, wherein the network is a wired or wireless network.

Embodiment 96. The method of embodiment 95, wherein the network is Ethernet, Bluetooth, or Wi-Fi.

Embodiment 97. The method of any one of embodiments 78-96, wherein the plurality of electrodes comprises an array of electrodes.

Embodiment 98. The method of embodiment 97, wherein the array of electrodes is embedded in an article.

Embodiment 99. The method of embodiment 98, wherein the article is a textile.

Embodiment 100. The method of embodiment 99, wherein the textile is incorporated into a wearable device.

Embodiment 101. The method of any one of embodiments 78-100, wherein the generating the spatial pattern of recommended neurostimulation impulses is performed by a processor.

Embodiment 102. The method of embodiment 101, wherein the control signal provides a set of instructions that, when executed by the processor, generates the spatial pattern of recommended neurostimulation impulses.

Embodiment 103. A system for providing neurostimulation, comprising: a telecommunications device; a stimulation module; an electrode array; a power source; the telecommunications device comprising: a software application, the software application configured to provide a user interface, the user interface configured to accept a gesture, wherein the gesture is physical contact with the user interface, and generate a control signal based at least in part on the gesture; a transmitter configured to transmit the control signal to the stimulation module; the electrode array comprising: a plurality of electrodes, wherein each electrode is electrically coupled to the power source via a switch, wherein each switch is configured to connect or disconnect a corresponding electrode to or from the power source; the stimulation module comprising: a receiver configured to receive the control signal; a processor operably coupled to the receiver, the processor configured to generate a pattern of recommended neurostimulation impulses from the control signal; and a switching unit configured to configure each switch to connect or disconnect the corresponding electrode responsive to the pattern of recommended neurostimulation impulses.

Embodiment 104. A system, comprising: a first device as in embodiment 1; and a second device as in embodiment 1.

Embodiment 105. The system of embodiment 105, wherein the second device is operably coupled to the neck of a subject, wherein the second device is configured to provide second neurostimulation to the spinal cord.

Embodiment 106. The system of embodiment 106, wherein the second device is configured to provide the second neurostimulation responsive to first neurostimulation provided by the first device.

Embodiment 107. The system of embodiment 107, wherein the second device is configured to provide the second neurostimulation simultaneously to the first neurostimulation provided by the first device.

Embodiment 108. The system of embodiment 107, wherein the second device is configured to provide the second neurostimulation after the first neurostimulation provided by the first device.

Embodiment 109. The system of any one of embodiments 107-109, wherein the second device is configured to provide the second neurostimulation for a longer duration than a duration of the first neurostimulation provided by the first device.

Embodiment 110. The system of any one of embodiments 107-110, wherein the second device is configured to provide the second neurostimulation for a same duration as a duration of the first neurostimulation provided by the first device.

Embodiment 111. A device comprising: a first article, wherein the first article comprises 1) a plurality of electrodes; and 2) a layer of hydrogel; and a second article, wherein the second article comprises 1) a circuit board operably connected to the plurality of electrodes; 2) a second top surface, wherein the second top surface comprises a plurality of visualization aids, wherein each visualization aid of the plurality of visualization aids independently corresponds to one of the plurality of electrodes; wherein when the first article and the second article are operably connected, the first article and the second article together form a wearable, wherein the wearable has a wearable size and a wearable shape adapted to fit on a human body part.

The invention claimed is:

1-90. (canceled)

91. A system comprising:
a first article, wherein the first article is flexible and comprises:
a first top surface, wherein the first top surface comprises a set of first connectors; and
a first bottom surface, wherein the first bottom surface comprises a plurality of electrodes,
wherein a number of first connectors of the set of first connectors is the same number as a number of electrodes of the plurality of electrodes, and
wherein each first connector of the set of first connectors is co-located with a corresponding electrode of the plurality of electrodes; and
a second article, wherein the second article is flexible and comprises:
a flexible circuit board operably connectable to the plurality of electrodes of the first article;
a second top surface; and
a second bottom surface, wherein the second bottom surface comprises a set of second connectors,
wherein the number of first connectors of the set of first connectors is the same number as a number of second connectors of the set of second connectors, and
wherein each first connector of the set of first connectors is independently configured to couple to one second connector of the set of second connectors,
wherein the set of first connectors and the set of second connectors are configured to form connections that hold the first article and the second article together and that operably connect the flexible circuit board to the plurality of electrodes when the set of first connectors is coupled to the set of second connectors,
wherein when the first article and the second article are operably connected, the first article and the second article together form a wearable, wherein the wearable has a wearable size and a wearable shape adapted to fit on a human body part.

92. The system of claim 91, wherein the second top surface comprises a plurality of visual indicators, wherein each visual indicator of the plurality of visual indicators independently corresponds to one of the plurality of electrodes, wherein the plurality of visual indicators are lights.

93. The system of claim 92, wherein each of the plurality of visual indicators is configured to provide a visible signal when an electrode corresponding to the visual indicator emits an electrical signal.

94. The system of claim 91, wherein each connector of the set of first connectors forms a magnetic connection with a corresponding connector of the set of second connectors,
wherein each of the magnetic connections is conductive.

95. The system of claim 91, wherein the second article is durable and reusable.

96. The system of claim 95, wherein the first article is configured for contact with human skin and is disposable.

97. The system of claim 91, further comprising:
a stimulator operably connectable to the second article, wherein the stimulator comprises:
a power supply, wherein the power supply comprises at least one of: a battery, or an alternating current (AC) adapter;
a processor operably connected to the power supply and configured to operate the system; and
stimulation circuitry operably connected to the processor, operably connected to the power supply, and configured to provide electrical stimulation signals to the flexible circuit board.

98. The system of claim 97, wherein the stimulator further comprises a wireless receiver operably connected to the processor and configured to receive instructions from a user, wherein the stimulator provides the electrical stimulation signals to the flexible circuit board based on the instructions from the user,
wherein the system is operated remotely by the user or locally by the user.

99. The system of claim 97, wherein the stimulator further comprises a motion sensor operably connected to the processor and configured to detect motion of a bodily portion.

100. The system of claim 99, further comprising non-transitory, computer-executable code which, when executed, is configured to track motion of the bodily portion in response to the electrical stimulation and to design a physical therapy regimen based at least in part on the motion of the bodily portion in response to the electrical stimulation, wherein the physical therapy regimen comprises a pattern of electrical signals applied via the plurality of electrodes.

101. The system of claim 100, wherein the wearable is configured to be adapted to a forearm,
wherein the stimulator is configured to be worn on a wrist,
wherein the motion sensor of the stimulator is configured to track motion of the wrist, and
wherein the non-transitory, computer-executable code, when executed, is configured to track motion of the wrist in response to the electrical stimulation of the forearm and to design a physical therapy regimen based at least in part on the motion of the wrist in response to the electrical stimulation of the forearm.

102. The system of claim 97, wherein the second article further comprises:
a plurality of switches, each switch independently addressable and comprising a first terminal and a second terminal,
wherein for each switch of the plurality of switches:
the first terminal of the respective switch is electrically coupled to the power supply,
the second terminal of the respective switch is electrically coupled to a corresponding connector of the set of second connectors, and
the respective switch is configured, when power is provided by the power supply to the first terminal of the respective switch, to provide electrical power to the corresponding connector.

103. The system of claim 102, wherein in the first article, the plurality of electrodes is sequentially connected in a daisy-chain configuration, and
wherein when the first article and the second article are operably connected, the plurality of switches is configured to be independently addressable to selectively provide power to any of the plurality of electrodes.

104. The system of claim 91, further comprising non-transitory, computer-executable code which, when executed, is configured to provide analysis of a performance of a human subject using the wearable in therapy and to determine a proposed therapy regimen based on the analysis, wherein the proposed therapy regimen comprises activation of the plurality of electrodes in a pattern that is determined to be likely to provide a physical therapy improvement based on the analysis.

105. The system of claim 104, wherein the analysis determines that the human subject cannot effectively grip an object.

106. The system of claim 91, further comprising non-transitory, computer-executable code which, when executed, is configured to cause a touch screen to display a grid, wherein the grid corresponds to a layout of the plurality of electrodes, wherein a touch of the touch screen on a point on the grid activates an electrode of the plurality of electrodes that corresponds to the point on the grid.

107. The system of claim 91, further comprising non-transitory, computer-executable code which, when executed, is configured to cause the system at least to perform:
a) receiving an input indicating a gesture of a user;
b) generating, based on the gesture of the user, a pattern of electrical stimulation for the human body part;
b) generating a control signal based on the pattern of electrical stimulation; and
d) providing electrical current to at least one of a plurality of electrodes based on the control signal to provide the pattern of electrical stimulation to the human body part.

108. The system of claim 91, wherein the first article further comprises a return electrode configured as a frame around the plurality of electrodes.
